(19) United States
(12) Patent Application Publication
(10) Pub. No.: US 2016/0333073 A1
(43) Pub. Date: Nov. 17, 2016

(54) DRUG DELIVERY SYSTEM

(71) Applicant: UCL BUSINESS PLC, London (GB)

(72) Inventors: David SHIMA, Hertfordshire (GB); Owen ANDERSON, London (GB)

(73) Assignee: UCL Business PLC, London (GB)

(21) Appl. No.: 15/113,091

(22) PCT Filed: Jan. 21, 2015
(86) PCT No.: PCT/GB2015/050135

§ 371 Date: Jul. 21, 2016

(30) Foreign Application Priority Data
Jan. 21, 2014 (GB) 1400994.8

(51) Int. Cl. C07K 14/705; C07K 16/24; G01N 33/68; A61K 47/48

(52) U.S. Cl. CPC C07K 14/70596; A61K 47/48246; A61K 47/48276; C07K 16/245; G01N 33/6872; G01N 2400/40; G01N 2333/70596

(57) ABSTRACT

The present invention concerns peptides comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, and wherein the N-terminus of the peptide comprises a D-amino acid and/or includes a protecting group, collagen or hyaluronic acid conjugates comprising the same peptides and a therapeutic or diagnostic agent, and compositions and uses thereof. It also concerns peptides comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, for use in the treatment or prevention of ocular diseases or conditions. Furthermore, it relates to a method of detecting a hyaluronic acid binding substance, the method comprising providing a sample of hyaluronic acid, contacting the sample of hyaluronic acid with a test substance, and detecting the presence of binding between the test substance and the hyaluronic acid.

The present invention relates to a drug delivery system. In particular, though not exclusively, it concerns collagen and/or hyaluronic acid binding conjugates as a depot drug delivery system.

The delivery of drugs into the eye via injection directly into the vitreous humour (‘vitreous’) has revolutionised the management of a number of debilitating ocular conditions, such as age-related macular degeneration (AMD), diabetic retinopathy, retinal vein occlusion, uveitis, and glaucoma. A major breakthrough, for example, has come with the development of vascular endothelial growth factor (VEGF) inhibitors. These drugs are usually administered via intravitreal injection, as they are poorly absorbed via other modes of delivery. However, this administration often needs to be repeated many times as the diseases for which VEGF inhibitors are required are often chronic in nature. Nevertheless, VEGF inhibitors can preserve or even improve vision, where previously the eye would have become at least partially blind.

There is a strong interest in developing the next generation of drugs to treat ocular conditions. However, a significant limitation of the utility of any drugs for treating such conditions is their half-life in the eye, since they rapidly diffuse from the vitreous, through the retina and into the choroidal space for clearance. Even large therapeutic agents, such as antibodies, have a 2-4 day half-life in the vitreous, which translates into 9-12 injections per year in order to provide maximum therapeutic efficacy. For smaller molecules, of which there are numerous in development, residence time in the vitreous is of the order of hours.

Frequent intravitreal injections are expensive and time consuming for the patient and healthcare provider. They also pose a small but significant risk of injection-related complications, such as infection or retinal detachment, which may threaten the quality of the patients' vision. With repeated injections the cumulative risk increases.

Therefore, there is increased interest in methods of prolonging the actions of intravitreally-administered drugs. Such methods would serve not only to reduce the frequency of injections (and subsequently the frequency of complications) but also to reduce the burden of provision for the health service. They would also help to reduce high concentration exposures of drugs to the retina, since VEGF antagonists have, for example, been shown to exacerbate retinal neural loss in such circumstances.

There are a number of methods under investigation (Anderson et al. Delivery of anti-angiogenic molecular therapies for retinal disease. Drug Discov Today. 2010 April; 15(7-8): 272-282). These include sequestering the active agent in a matrix or an implant, in order to slow down its release into the vitreous. These may be biodegradable or non-biodegradable. Implants may be injected directly into the vitreous or surgically implanted into the vitreous (Haller et al. Randomized, sham-controlled trial of dexamethasone intravitreal implant in patients with macular edema due to retinal vein occlusion. Ophthalmology 2010; 117(6): 1134-1146, and Pavesio et al. Evaluation of an intravitreal fluocinolone acetonide implant versus standard systemic therapy in noninfectious posterior uveitis. Ophthalmology 2010; 117(3): 567-575). Another technique under development is the endogenous production of pharmacologically active molecules through viral gene transfection (Bainbridge et al. Effect of gene therapy on visual function in Leber's congenital amaurosis. N. Engl. J. Med. 2008; 358: 2231-2239). However, control of gene expression is subsequently required so that pharmacologically appropriate levels of therapeutic agent may be achieved.

It has been shown that intravitreal delivery of steroid agents may be achieved using reservoir depot devices, either surgically implanted (e.g. Retisert™, Bausch & Lomb, Inc.) or injected (e.g. Iluvien™, Alimera Sciences/pSivida, Inc.). However, specific injection devices or surgical procedures are required for delivering such agents into the eye. Furthermore, these agents (Retisert™ and Iluvien™) are non-biodegradable, and non-therapeutic components remain in the eye for a prolonged period of time. Moreover, the devices employed for these agents are limited in terms of payload, and their use is therefore restricted to very potent molecules such as steroids. Biodegradable particles, such as poly lactic-co-glycolic acid (PLGA) microspheres, are in development, but currently suffer from certain drawbacks in relation to payload, incompatibility due to organic solvents used in their manufacture, and interference with patients' vision.

The use of binding molecules to influence the pharmacokinetic properties of drugs has been investigated outside the field of ophthalmology. One example of this involves the use of peptides which have been designed to bind to particular targets, such as albumin. Albumin is a relatively large molecule, which is not filtered via the kidney glomerulus, and hence is retained in the blood circulation. Drugs conjugated to albumin binding peptides are retained in the circulation for longer periods of time, as compared to non-conjugated drugs. As a result, they can be administered less frequently.

One such binding moiety, which has been shown to exhibit effective binding affinity to hyaluronic acid, is HABP35, a short peptide derived from the mouse RHAMM receptor (receptor for hyaluronan-mediated motility). The peptide is made up of the hyaluronic acid (HA) binding domain I sequence followed by the mouse HA binding domain II sequence, and its sequence was determined from a publicly available source. The RHAMM receptor has previously been studied in the fields of oncology, immunology and angiogenesis, while HABP35 has been specifically studied due to its effect on wound infections (Zaleski et al. Hyaluronic acid binding peptides prevent experimental staphylococcal wound infection. Antimicrob Agents Chemother. 2006; 50(11); 3856-3860). However, such binding molecules, with regard to drug delivery, have not been investigated in the fields of ophthalmology, dermatology or arthrology.

Accordingly, in an embodiment of the invention, there is provided an isolated peptide comprising at least one motif having the amino acid sequence B¹-X₃₋₁₀-B², wherein B¹ and B² are identical or different and each is a basic amino acid (e.g. lysine or arginine) and X₃₋₁₀ is a sequence of 3 to 10 identical or different non-acidic amino acids, and wherein the N-terminus of the peptide comprises a D-amino acid and/or includes a protecting group. Preferably, the peptide has two or three motifs B¹-X₃₋₁₀-B², more preferably four. Such motifs may be arranged in a contiguous, sequential or overlapping manner. Preferably, such motifs are overlapping. Furthermore, preferable motfis have the structure B¹-X₅₋₁₀-B² or B¹-X₆₋₈-B², or B¹-X₆-B², B¹-X₇-B², or B¹-X₈-B², most preferably B¹-X₇-B² (i.e. where B¹ and B² are separated by a sequence of 7 identical or different non-acidic amino acids).

In a preferred embodiment, the peptide has a sequence with at least 60% homology to SEQ ID No. 1, or a functional portion/fragment thereof.

The amino acid sequence according to SEQ ID No. 1 (also known as HABP35) relates to the mouse receptor for hyaluronan mediated motility (RHAMM), which comprises the mouse RHAMM hyaluronic acid binding domain I sequence followed by the mouse RHAMM hyaluronic acid binding domain II, separated by a linker (i.e. VVV). The specific amino acid sequence of SEQ ID No. 1 is LKQKIKHVVKLKVVVKLRSQLVKRKQN.

Since the two main components of the vitreous are collagen and hyaluronic acid, the present invention provides binding conjugates which have the ability to bind to collagen and/or hyaluronic acid, and thereby act as anchoring substrates to which active therapeutic or diagnostic agents may be reversibly attached. In addition, given that collagen and hyaluronic acid are also abundant components of connective, epithelial and neural tissues, such binding conjugates have significant applications in the treatment of a range of arthrological and dermatological conditions, as well as a range of ocular conditions.

For example, by linking drugs, such as VEGF inhibitors, antibodies or novel targeted small molecules, to conjugates that bind to constituents in the vitreous, primarily collagen and/or hyaluronic acid, the drugs' rate of clearance from the vitreous is reduced, and thereby released over a longer period of time. Increasing the drug half-life in the eye means prolonged drug delivery to the retina. This has both financial rewards (reduced number of hospital visits for injections) and patient safety rewards (reduced number of injections means reduced risk of an injection related complication). Currently there are no depot delivery devices in clinical practice for the delivery of VEGF inhibitors (e.g. ranibizumab, pegaptanib, bevacizumab, and aflibercept).

Surprisingly, it has been found that these sequences of amino acids provide the peptide with a reversible affinity to the chemical structures of collagen and/or hyaluronic acid, such as found in fibrous, connective, epithelial, and neural tissues, as well as in the vitreous humour of the eye. In addition, the presence of a D-amino acid and/or the inclusion of a protecting group at the N-terminus can potentially provide the peptide with improved stability against enzymatic degradation, e.g. in vivo.

In another embodiment of the invention, there is provided a collagen or hyaluronic acid binding conjugate comprising a peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, wherein the N-terminus of the peptide comprises a D-amino acid and/or includes a protecting group, and a therapeutic or diagnostic agent, wherein the therapeutic or diagnostic agent is optionally bound to the peptide by means of a linker. It has been advantageously found that the binding action of the conjugate significantly slows down the removal rate (and thus excretion rate) of the therapeutic or diagnostic agent. In one aspect, the conjugate acts as a depot drug delivery system.

Preferably, the collagen or hyaluronic acid binding conjugate comprises a peptide having a sequence with at least 60% homology to SEQ ID No. 1, or a functional portion or fragment thereof, and a therapeutic or diagnostic agent, wherein the therapeutic or diagnostic agent is optionally bound to the peptide by means of a linker.

The protecting group according to the invention refers to protection of the α-amino group of the N-terminus amino acid. Suitable protecting groups include those selected from the group consiting of acetyl, benzoyl, benzyl, tert-butoxycarbonyl, carbobenzyloxy, p-methoxybenzyl carbonyl, p-methoxybenzyl, 9-fluorenylmethyloxycarbonyl, 3,4-dimethoxybenzyl, p-methoxyphenyl, tosyl, and nosyl. Preferably, the protecting group is acetyl, benzoyl, benzyl, tert-butoxycarbonyl, carbobenzyloxy, p-methoxybenyl carbonyl, p-methoxybenzyl, 3,4-dimethoxybenzyl, or p-methoxyphenyl. Most preferably, the protecting group is acetyl.

As used herein, the term “collagen” refers to a group of naturally occurring proteins found in humans and animals, especially in the flesh and connective tissues. In the form of elongated fibrils, it is mostly found in fibrous tissues such as tendon, ligament and skin, and is also abundant in cornea, cartilage, bone, blood vessels, the gut, and intervertebral disc.

The term “hyaluronic acid (HA)” refers to an anionic, nonsulfated glycosaminoglycan distributed widely throughout connective, epithelial, and neural tissues. It is also found in the vitreous humour. It is one of the main components of the extracellular matrix, and contributes significantly to cell proliferation and migration, and may also be involved in the progression of some malignant tumours. The term may be used synonymously with the terms “hyaluronan” and “hyaluronate”. It is a linear non-branching molecule made up of repeating units of D-glucuronic acid and D-N-acetyl-glucosamine, as shown below.

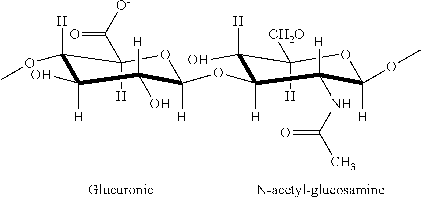

The term “vitreous” refers to the transparent, colourless gel that fills the space between the lens and retina lining the back of the eyeball of humans and other vertebrates. This term can be used synonymously with the terms “vitreous humour” and “vitreous body”.

The term “cartilage” refers to the flexible connective tissue found in many areas of the human or animal body, including the joints between bones, the rib cage, the ear, the nose, the bronchial tubes and the intervertebral discs. This tissue is not as hard and rigid as bone but is stiffer and less flexible than muscle. It is composed of specialised cells called chondrocytes that produce a large amount of extracellular matrix composed of collagen fibres, abundant ground substance rich in proteoglycan, and elastin fibres. Cartilage is classified in three types, elastic cartilage, hyaline cartilage, and fibrocartilage, which differ in the relative amounts of these three main components.

The present invention relates to a drug delivery system, in which collagen and/or hyaluronic acid binding conjugates may be employed to target and reversibly attach therapeutic or diagnostic agents to a specific site for treatment. The result of this attachment is such that the therapeutic or diagnostic agents are not so readily removed from the treatment site and thus have a longer residence time in which to exert their effect.

Surprisingly, it has been found that this may be achieved using an isolated peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, and wherein the N-terminus of the peptide comprises a D-amino acid and/or includes a protecting group, preferably a peptide having a protein sequence with at least 60% homology to SEQ ID No. 1, or a functional portion or fragment thereof. The precise sequence of SEQ ID No. 1, however, is known in the art and does not form part of the claimed subject matter as a peptide per se. Nevertheless, its utility as a drug delivery system has not been previously contemplated in certain circumstances.

Functional fragments and portions of the peptide include those fragments and portions that maintain one or more functions of the parent peptide. It is recognised that the gene for cDNA encoding a peptide may be considerably mutated without materially altering one or more of the peptides functions. First, the generic code is well-known to be degenerate, and thus different codons encode the same amino acids. Second, even where an amino acid substitution is introduced, the mutation may be conservative and have no material impact on the essential functions of the protein. Third, part of a peptide chain may be deleted without impairing or eliminating all of its functions. Fourth, insertions or deletions may be made in the peptide chain, for example, adding epitope tags, without impairing or eliminating its functions. Functional fragments and portions also include those in which a function is enhanced.

Other modifications that may be made without materially impairing one or more functions of the peptide include, for example, in vivo or in vitro chemical and biochemical modifications or which incorporate unusual amino acids. Such modifications include, for example, acetylation, carboxylation, phosphorylation, glycosylation, ubiquination, labelling, such as with radionuclides (e.g. ³²P), and various enzymatic modifications.

Peptides may be branched as a result of such modifications, and they may be cyclic, with or without branching. Cyclic, branched and branched cyclic peptides may result from post-translation natural processes or may be made by synthetic methods.

In embodiments, the C-terminus of the peptide, or the functional fragment of portion thereof, may be converted to an amide. In particular, with regard to functional fragments and portions of the peptide, this avoids the unnatural introduction of a charged group at a site in the peptide, where the same site in the parent peptide would have no such charge. Furthermore, it means that the peptide may be more likely to be recognised as if it were part of the whole protein from which is was chosen. In addition or alternatively, the presence of such functionality at the C-terminus may provide greater resistance to the breakdown resulting from the action of exopeptidases.

Protein homologues of the present invention are typically characterised by possession of at least 60%, such as at least 70%, 80%, 90%, 95%, or even 98% sequence homology, counted over the full length alignment with the amino acid sequence using NCBI Basic Protein Blast 2.0. Preferably, the isolated peptide has a protein sequence with at least 80% homology, even more preferably 90% homology, most preferably 95% homology to SEQ ID No. 1, or is a functional portion or fragment thereof. Preferably, the term “homology” as used herein, refers to the presence of identical amino acids or amino acids of the same chemical class, e.g. polar, basic, acidic, hydrophobic amino acid types. The characterisation of amino acid types is well known to the skilled person.

In a preferred embodiment, protein homologues of the present invention are typically characterised by possession of at least 60%, such as at least 70%, 80%, 90%, 95%, or even 98% sequence identity, counted over the full length alignment with the amino acid sequence using NCBI Basic Protein Blast 2.0. Preferably, the isolated peptide has a protein sequence with at least 80% identity, even more preferably 90% identity, most preferably 95% identity to SEQ ID No. 1, or is a functional portion or fragment thereof.

In terms of functional fragments or portions of SEQ ID No. 1, the peptide of the invention may comprise at least 5 contiguous amino acids from SEQ ID No. 1 provided that such fragments or portions possess at least 70% of the affinity of the peptide having at least 60% homology to SEQ ID No. 1 to hyaluronic acid and/or at least 70% of the affinity of the peptide having at least 60% homology to SEQ ID No. 1 to collagen. In a preferable aspect, the peptide comprises at least 6, 7, 8, or 9 contiguous amino acids, more preferably at least 10, 11, or 12 contiguous amino acids, and shows at least 70% of the affinity of the peptide having at least 60% homology to SEQ ID No. 1 to hyaluronic acid and/or at least 70% of the affinity of the peptide having at least 60% homology to SEQ ID No. 1 to collagen. Functional fragments and portions of the peptide contain at least one sequence of amino acids with the motif B¹-X₃₋₁₀-B², preferably two or three.

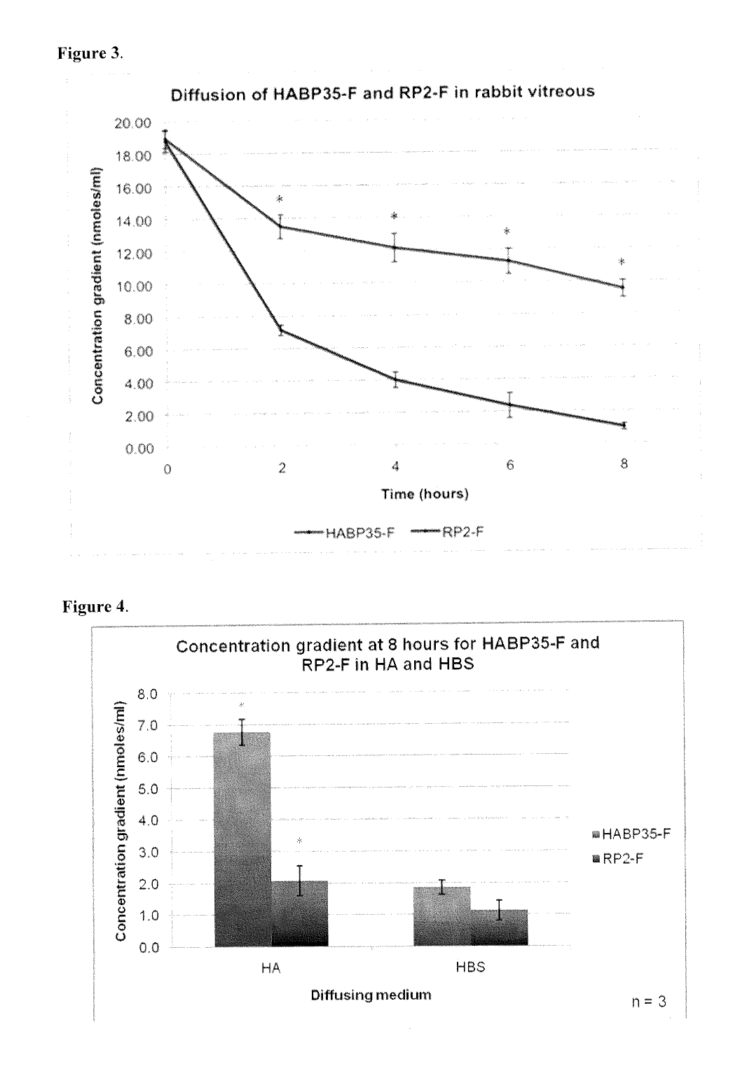

The affinity of the peptide may be defined in terms of its binding affinity to hyaluronic acid and/or collagen, and assessed by way of its diffusion from one chamber of a micro-equilibrium dialyser containing vitreous matter (e.g. hyaluronic acid) to another chamber containing vitreous matter in the absence of such a binding peptide. Thus, the concentration of peptide remaining in the initial chamber over time provides a quantitative parameter for assessing the amount of peptide remaining in the vitreous, this parameter being innately governed by peptide binding properties to hyaluronic acid. A comparison with the binding properties of the peptide of SEQ ID No. 1 allows the relative affinity to the determined (for example, see FIG. 3).

The affinity of the peptide according to the invention, or a functional portion or fragment thereof, is at least 70% of the affinity of the peptide having at least 60% homology to SEQ ID No. 1 to hyaluronic acid and/or at least 70% of the affinity of the peptide having at least 60% homology to SEQ ID No. 1 to collagen. The affinity may be stronger than this nevertheless and levels of affinity such as at least 75%, 80%, and 95% can be mentioned. In a preferable embodiment, the affinity of the peptide is at least 85% of the affinity of the peptide having at least 60% homology to SEQ ID No. 1 to hyaluronic acid and/or at least 85% of the affinity of the peptide having at least 60% homology to SEQ ID No. 1 to collagen, more preferably at least 90%, even more preferably at least 95% or 97%.

Specific fragments or portions of SEQ ID No. 1 that can be mentioned include those listed in Table 1. The N-terminus of these peptides may comprise a D-amino acid and/or include a protecting group, and the C-terminus may be converted to an amide.

TABLE 1

| Sequence Identifier | Peptide Sequence |
|---|---|
| SEQ ID No. 2 | LKQKIKHVVKLKVVVKLRSQLVKRKQ |
| SEQ ID No. 3 | LKQKIKHVVKLKVVVKLRSQLVKRK |
| SEQ ID No. 4 | LKQKIKHVVKLKVVVKLRSQLVKR |
| SEQ ID No. 5 | LKQKIKHVVKLKVVVKLRSQLVK |
| SEQ ID No. 6 | LKQKIKHVVKLKVVVKLRSQLV |
| SEQ ID No. 7 | LKQKIKHVVKLKVVVKLRSQL |
| SEQ ID No. 8 | LKQKIKHVVKLKVVVKLRSQ |
| SEQ ID No. 9 | LKQKIKHVVKLKVVVKLRS |
| SEQ ID No. 10 | LKQKIKHVVKLKVVVKLR |
| SEQ ID No. 11 | LKQKIKHVVKLKVVVKL |
| SEQ ID No. 12 | LKQKIKHVVKLKVVVK |
| SEQ ID No. 13 | LKQKIKHVVKLKVVV |
| SEQ ID No. 14 | LKQKIKHVVKLKVV |
| SEQ ID No. 15 | LKQKIKHVVKLKV |
| SEQ ID No. 16 | LKQKIKHVVKLK |
| SEQ ID No. 17 | LKQKIKHVVKL |
| SEQ ID No. 18 | LKQKIKHVVK |
| SEQ ID No. 19 | LKQKIKHVV |
| SEQ ID No. 20 | LKQKIKHV |
| SEQ ID No. 21 | LKQKIKH |
| SEQ ID No. 22 | LKQKIK |
| SEQ ID No. 23 | KQKIKHVVKLKVVVKLRSQLVKRKQN |
| SEQ ID No. 24 | QKIKHVVKLKVVVKLRSQLVKRKQN |
| SEQ ID No. 25 | KIKHVVKLKVVVKLRSQLVKRKQN |
| SEQ ID No. 26 | IKHVVKLKVVVKLRSQLVKRKQN |
| SEQ ID No. 27 | KHVVKLKVVVKLRSQLVKRKQN |
| SEQ ID No. 28 | HVVKLKVVVKLRSQLVKRKQN |
| SEQ ID No. 29 | VVKLKVVVKLRSQLVKRKQN |
| SEQ ID No. 30 | VKLKVVVKLRSQLVKRKQN |
| SEQ ID No. 31 | KLKVVVKLRSQLVKRKQN |
| SEQ ID No. 32 | LKVVVKLRSQLVKRKQN |
| SEQ ID No. 33 | KVVVKLRSQLVKRKQN |
| SEQ ID No. 34 | VVVKLRSQLVKRKQN |
| SEQ ID No. 35 | VVKLRSQLVKRKQN |
| SEQ ID No. 36 | VKLRSQLVKRKQN |
| SEQ ID No. 37 | KLRSQLVKRKQN |
| SEQ ID No. 38 | LRSQLVKRKQN |
| SEQ ID No. 39 | RSQLVKRKQN |
| SEQ ID No. 40 | KQKIKHVVKLKVVVKLRSQLVKRKQ |
| SEQ ID No. 41 | QKIKHVVKLKVVVKLRSQLVKRK |
| SEQ ID No. 42 | KIKHVVKLKVVVKLRSQLVKR |
| SEQ ID No. 43 | IKHVVKLKVVVKLRSQLVK |
| SEQ ID No. 44 | KHVVKLKVVVKLRSQLV |
| SEQ ID No. 45 | HVVKLKVVVKLRSQL |
| SEQ ID No. 46 | VVKLKVVVKLRSQ |
| SEQ ID No. 47 | VKLKVVVKLRS |
| SEQ ID No. 48 | KLKVVVKLR |
| SEQ ID No. 49 | LKVVVKL |
| SEQ ID No. 50 | KVVVK |
| SEQ ID No. 51 | QKIKHVVKLKVVVKLRSQLVKRKQ |
| SEQ ID No. 52 | KIKHVVKLKVVVKLRSQLVKRKQ |
| SEQ ID No. 53 | IKHVVKLKVVVKLRSQLVKRKQ |
| SEQ ID No. 54 | KHVVKLKVVVKLRSQLVKRKQ |
| SEQ ID No. 55 | HVVKLKVVVKLRSQLVKRKQ |
| SEQ ID No. 56 | VVKLKVVVKLRSQLVKRKQ |
| SEQ ID No. 57 | VKLKVVVKLRSQLVKRKQ |
| SEQ ID No. 58 | KLKVVVKLRSQLVKRKQ |
| SEQ ID No. 59 | LKVVVKLRSQLVKRKQ |
| SEQ ID No. 60 | KVVVKLRSQLVKRKQ |
| SEQ ID No. 61 | VVVKLRSQLVKRKQ |
| SEQ ID No. 62 | VVKLRSQLVKRKQ |
| SEQ ID No. 63 | VKLRSQLVKRKQ |
| SEQ ID No. 64 | KLRSQLVKRKQ |
| SEQ ID No. 65 | LRSQLVKRKQ |
| SEQ ID No. 66 | RSQLVKRKQ |
| SEQ ID No. 67 | QKIKHVVKLKVVVKLRSQLVKRK |
| SEQ ID No. 68 | KIKHVVKLKVVVKLRSQLVKRK |
| SEQ ID No. 69 | IKHVVKLKVVVKLRSQLVKRK |
| SEQ ID No. 70 | KHVVKLKVVVKLRSQLVKRK |
| SEQ ID No. 71 | HVVKLKVVVKLRSQLVKRK |
| SEQ ID No. 72 | VVKLKVVVKLRSQLVKRK |
| SEQ ID No. 73 | VKLKVVVKLRSQLVKRK |
| SEQ ID No. 74 | KLKVVVKLRSQLVKRK |
| SEQ ID No. 75 | LKVVVKLRSQLVKRK |
| SEQ ID No. 76 | KVVVKLRSQLVKRK |
| SEQ ID No. 77 | VVVKLRSQLVKRK |
| SEQ ID No. 78 | VVKLRSQLVKRK |
| SEQ ID No. 79 | VKLRSQLVKRK |
| SEQ ID No. 80 | KLRSQLVKRK |
| SEQ ID No. 81 | LRSQLVKRK |
| SEQ ID No. 82 | RSQLVKRK |
| SEQ ID No. 83 | QKIKHVVKLKVVVKLRSQLVKR |
| SEQ ID No. 84 | KIKHVVKLKVVVKLRSQLVKR |
| SEQ ID No. 85 | IKHVVKLKVVVKLRSQLVKR |
| SEQ ID No. 86 | KHVVKLKVVVKLRSQLVKR |
| SEQ ID No. 87 | HVVKLKVVVKLRSQLVKR |
| SEQ ID No. 88 | VVKLKVVVKLRSQLVKR |
| SEQ ID No. 89 | VKLKVVVKLRSQLVKR |
| SEQ ID No. 90 | KLKVVVKLRSQLVKR |
| SEQ ID No. 91 | LKVVVKLRSQLVKR |
| SEQ ID No. 92 | KVVVKLRSQLVKR |
| SEQ ID No. 93 | VVVKLRSQLVKR |
| SEQ ID No. 94 | VVKLRSQLVKR |
| SEQ ID No. 95 | VKLRSQLVKR |
| SEQ ID No. 96 | KLRSQLVKR |
| SEQ ID No. 97 | LRSQLVKR |
| SEQ ID No. 98 | RSQLVKR |
| SEQ ID No. 99 | QKIKHVVKLKVVVKLRSQLVK |
| SEQ ID No. 100 | KIKHVVKLKVVVKLRSQLVK |
| SEQ ID No. 101 | IKHVVKLKVVVKLRSQLVK |
| SEQ ID No. 102 | KHVVKLKVVVKLRSQLVK |
| SEQ ID No. 103 | HVVKLKVVVKLRSQLVK |
| SEQ ID No. 104 | VVKLKVVVKLRSQLVK |
| SEQ ID No. 105 | VKLKVVVKLRSQLVK |
| SEQ ID No. 106 | KLKVVVKLRSQLVK |
| SEQ ID No. 107 | LKVVVKLRSQLVK |
| SEQ ID No. 108 | KVVVKLRSQLVK |
| SEQ ID No. 109 | VVVKLRSQLVK |
| SEQ ID No. 110 | VVKLRSQLVK |
| SEQ ID No. 111 | VKLRSQLVK |
| SEQ ID No. 112 | KLRSQLVK |
| SEQ ID No. 113 | LRSQLVK |
| SEQ ID No. 114 | RSQLVK |
| SEQ ID No. 115 | QKIKHVVKLKVVVKLRSQLV |
| SEQ ID No. 116 | KIKHVVKLKVVVKLRSQLV |
| SEQ ID No. 117 | IKHVVKLKVVVKLRSQLV |
| SEQ ID No. 118 | KHVVKLKVVVKLRSQLV |
| SEQ ID No. 119 | HVVKLKVVVKLRSQLV |
| SEQ ID No. 120 | VVKLKVVVKLRSQLV |
| SEQ ID No. 121 | VKLKVVVKLRSQLV |
| SEQ ID No. 122 | KLKVVVKLRSQLV |
| SEQ ID No. 123 | LKVVVKLRSQLV |
| SEQ ID No. 124 | KVVVKLRSQLV |
| SEQ ID No. 125 | QKIKHVVKLKVVVKLRSQL |
| SEQ ID No. 126 | KIKHVVKLKVVVKLRSQL |
| SEQ ID No. 127 | IKHVVKLKVVVKLRSQL |
| SEQ ID No. 128 | KHVVKLKVVVKLRSQL |
| SEQ ID No. 129 | HVVKLKVVVKLRSQL |
| SEQ ID No. 130 | VVKLKVVVKLRSQL |
| SEQ ID No. 131 | VKLKVVVKLRSQL |
| SEQ ID No. 132 | KLKVVVKLRSQL |
| SEQ ID No. 133 | LKVVVKLRSQL |
| SEQ ID No. 134 | KVVVKLRSQL |
| SEQ ID No. 135 | QKIKHVVKLKVVVKLRSQ |
| SEQ ID No. 136 | KIKHVVKLKVVVKLRSQ |
| SEQ ID No. 137 | IKHVVKLKVVVKLRSQ |
| SEQ ID No. 138 | KHVVKLKVVVKLRSQ |
| SEQ ID No. 139 | HVVKLKVVVKLRSQ |
| SEQ ID No. 140 | VVKLKVVVKLRSQ |
| SEQ ID No. 141 | VKLKVVVKLRSQ |
| SEQ ID No. 142 | KLKVVVKLRSQ |
| SEQ ID No. 143 | LKVVVKLRSQ |
| SEQ ID No. 144 | KVVVKLRSQ |
| SEQ ID No. 145 | QKIKHVVKLKVVVKLRS |
| SEQ ID No. 146 | KIKHVVKLKVVVKLRS |
| SEQ ID No. 147 | IKHVVKLKVVVKLRS |
| SEQ ID No. 148 | KHVVKLKVVVKLRS |
| SEQ ID No. 149 | HVVKLKVVVKLRS |
| SEQ ID No. 150 | VVKLKVVVKLRS |
| SEQ ID No. 151 | VKLKVVVKLRS |
| SEQ ID No. 152 | KLKVVVKLRS |
| SEQ ID No. 153 | LKVVVKLRS |
| SEQ ID No. 154 | KVVVKLRS |
| SEQ ID No. 155 | QKIKHVVKLKVVVKLR |
| SEQ ID No. 156 | KIKHVVKLKVVVKLR |
| SEQ ID No. 157 | IKHVVKLKVVVKLR |
| SEQ ID No. 158 | KHVVKLKVVVKLR |
| SEQ ID No. 159 | HVVKLKVVVKLR |
| SEQ ID No. 160 | VVKLKVVVKLR |
| SEQ ID No. 161 | VKLKVVVKLR |
| SEQ ID No. 162 | KLKVVVKLR |
| SEQ ID No. 163 | LKVVVKLR |
| SEQ ID No. 164 | KVVVKLR |
| SEQ ID No. 165 | VVVKLR |
| SEQ ID No. 166 | VVKLR |
| SEQ ID No. 167 | QKIKHVVKLKVVVKL |
| SEQ ID No. 168 | KIKHVVKLKVVVKL |
| SEQ ID No. 169 | IKHVVKLKVVVKL |
| SEQ ID No. 170 | KHVVKLKVVVKL |
| SEQ ID No. 171 | HVVKLKVVVKL |
| SEQ ID No. 172 | VVKLKVVVKL |
| SEQ ID No. 173 | VKLKVVVKL |
| SEQ ID No. 174 | KLKVVVKL |
| SEQ ID No. 175 | LKVVVKL |
| SEQ ID No. 176 | KVVVKL |
| SEQ ID No. 177 | QKIKHVVKLKVVVK |
| SEQ ID No. 178 | KIKHVVKLKVVVK |
| SEQ ID No. 179 | IKHVVKLKVVVK |
| SEQ ID No. 180 | KHVVKLKVVVK |
| SEQ ID No. 181 | HVVKLKVVVK |
| SEQ ID No. 182 | VVKLKVVVK |
| SEQ ID No. 183 | VKLKVVVK |
| SEQ ID No. 184 | KLKVVVK |
| SEQ ID No. 185 | LKVVVK |
| SEQ ID No. 186 | QKIKHVVKLKVVV |
| SEQ ID No. 187 | KIKHVVKLKVVV |
| SEQ ID No. 188 | IKHVVKLKVVV |
| SEQ ID No. 189 | KHVVKLKVVV |
| SEQ ID No. 190 | HVVKLKVVV |
| SEQ ID No. 191 | QKIKHVVKLKVV |
| SEQ ID No. 192 | KIKHVVKLKVV |
| SEQ ID No. 193 | IKHVVKLKVV |
| SEQ ID No. 194 | KHVVKLKVV |
| SEQ ID No. 195 | HVVKLKVV |
| SEQ ID No. 196 | QKIKHVVKLKV |
| SEQ ID No. 197 | KIKHVVKLKV |
| SEQ ID No. 198 | IKHVVKLKV |
| SEQ ID No. 199 | KHVVKLKV |
| SEQ ID No. 200 | HVVKLKV |
| SEQ ID No. 201 | QKIKHVVKLK |
| SEQ ID No. 202 | KIKHVVKLK |
| SEQ ID No. 203 | IKHVVKLK |
| SEQ ID No. 204 | KHVVKLK |
| SEQ ID No. 205 | HVVKLK |
| SEQ ID No. 206 | QKIKHVVKL |
| SEQ ID No. 207 | KIKHVVKL |
| SEQ ID No. 208 | IKHVVKL |
| SEQ ID No. 209 | KHVVKL |
| SEQ ID No. 210 | QKIKHVVK |
| SEQ ID No. 211 | KIKHVVK |
| SEQ ID No. 212 | IKHVVK |
| SEQ ID No. 213 | KHVVK |
| SEQ ID No. 214 | KIKHVV |
| SEQ ID No. 215 | IKHVV |
| SEQ ID No. 216 | QKIKHV |
| SEQ ID No. 217 | KIKHV |
| SEQ ID No. 218 | QKIKH |
| SEQ ID No. 219 | LKQKIKHVVKLK |
| SEQ ID No. 220 | KQKIKHVVKLK |
| SEQ ID No. 221 | KQKIKHVVKL |
| SEQ ID No. 222 | KQKIKHVVK |
| SEQ ID No. 223 | KQKIKHVV |
| SEQ ID No. 224 | KQKIKHV |
| SEQ ID No. 225 | KQKIKH |
| SEQ ID No. 226 | KQKIK |
| SEQ ID No. 227 | QKIKHVVKLK |
| SEQ ID No. 228 | KIKHVVKLK |
| SEQ ID No. 229 | IKHVVKLK |
| SEQ ID No. 230 | KHVVKLK |
| SEQ ID No. 231 | HVVKLK |
| SEQ ID No. 232 | KLRSQLVKRKQN |
| SEQ ID No. 233 | KLRSQLVKRKQ |
| SEQ ID No. 234 | KLRSQLVKRK |
| SEQ ID No. 235 | KLRSQLVKR |
| SEQ ID No. 236 | KLRSQLVK |
| SEQ ID No. 237 | LRSQLVKRKQ |
| SEQ ID No. 238 | RSQLVKRKQ |

The peptide forming part of the invention is an isolated biological component in the sense that it has been substantially separated from other biological components in the cell of the organism in which the component may naturally occur, i.e. other chromosomal or extra-chromosomal DNA and RNA, proteins and organelles. Nucleic acids and proteins that have been isolated include nucleic acids and proteins purified by standard purification methods. The term “isolated” also embraces nucleic acids and proteins prepared by the recombinant expression in a host cell as well as chemically synthesised nucleic acids and proteins.

In the collagen or hyaluronic acid binding conjugate of the invention, the therapeutic or diagnostic agent may be covalently or non-covalently bound to the peptide.

When the therapeutic or diagnostic agent is non-covalently bound to the peptide, binding may be achieved by means of a biotin-streptavidin complex. For example, the peptide may be covalently bound to the biotin moiety, optionally via a linker, and the therapeutic or diagnostic agent may be covalently bound to the streptavidin moiety, optionally via a linker. Alternatively, the peptide may be covalently bound to the streptavidin moiety, optionally via a linker, and the therapeutic or diagnostic agent may be covalently bound to the biotin moiety, optionally via a linker. The optional linker group in this instance may be the same as the linker which optionally binds the peptide to the therapeutic or diagnostic agent.

The collagen or hyaluronic acid binding conjugate may contain a linker which binds the peptide to the therapeutic or diagnostic agent. Any commercially available cross-linker may be an appropriate linker. Such cross-linkers are typically linear molecules, which have chemically reactive groups at each end. Under appropriate conditions, these cross-linkers can form a covalent attachment between two molecules, i.e. the peptide and the therapeutic or diagnostic agent. Importantly, the cross-linker binds one end to the peptide and the other end to the therapeutic or diagnostic agent, while maintaining the biological function of each. Preferably, the linker of the invention, when present, is a heterobifunctional cross-linker having different reactive groups at each end. This allows more specific and sequential (two-step) conjugation, minimising the possibility of polymerization or dimerisation of like groups, e.g. therapeutic agent to therapeutic agent linking, or peptide to peptide linking.

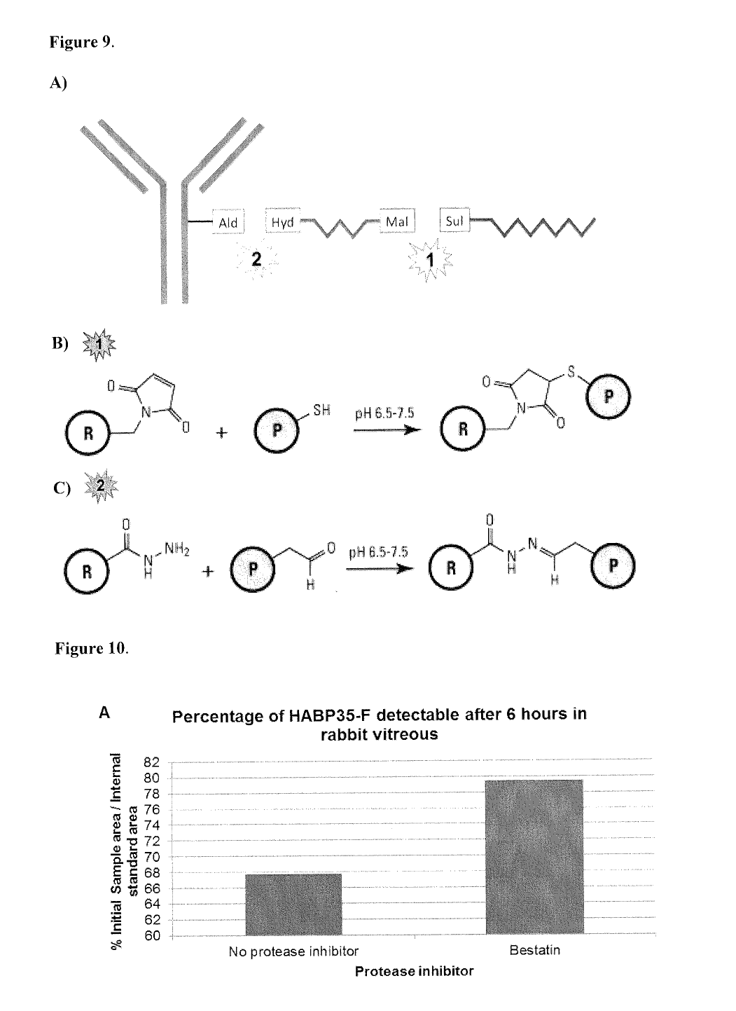

In particular, when present, the linker may comprise a short-chain peptide (e.g. of from 1 to 10 amino acids), a polyethylene glycol oligomer (e.g. of 2 to 10 polyethylene glycol units), a C₁₋₂₀ alkylene group (e.g. a C₁₋₁₀ alkylene group), a C₂₋₂₀ alkenylene group (e.g. a C₂₋₁₀ alkenylene group), maleimide and hydrazide functional groups separated by a C₁₋₁₀ alkylene group (e.g. a C₁₋₁₀ alkylene group) or C₂₋₁₀ alkenylene group (e.g. a C₂₋₁₀ alkenylene group) (e.g. see FIG. 9), or any combination thereof. Preferably, the linker comprises a short-chain peptide of from 1 to 10 amino acids, maleimide and hydrazide functional groups separated by a C₁₋₁₀ alkylene group, or any combination thereof. Such linkers prevent steric hindrance from occurring between the peptide and the therapeutic or diagnostic agent.

The term ‘C_{x-y} alkylene’ as used herein refers to a divalent hydrocarbon group (e.g. —CH₂— or >CCH₃) which is a linear or branched saturated hydrocarbon group containing from x to y carbon atoms. Examples of C₁₋₁₀ alkylene groups include methylene, ethylene, propylene, butylene, hexylene, etc.

The term ‘C_{x-y} alkenylene’ as used herein refers to a divalent hydrocarbon group (e.g. —CH═CH— or >C═CH₂) which is a linear or branched hydrocarbon group containing one or more carbon-carbon double bonds and having from x to y carbon atoms. Examples of C₂₋₁₀ alkenylene groups include vinylene, propenylene, butenylene, hexenylene, etc.

The polyethylene glycol oligomer which may form part of the linker refers to an oligomer having from 2 to 10 repeating units of ethylene oxide, i.e. of the formula H—(O—CH₂—CH₂)ₙ—OH where n is an integer from 2 to 10.

Preferably, the short-chain peptide comprises the amino acids glycine, serine, lysine, cysteine, glutamic acid and/or aspartic acid, such as -GGGS-, GGGSK, GGGSKC, etc. In addition, the linker, when present, is preferably located at the C-terminus of the peptide.

The linker may also further comprise a labelling moiety. Suitable labelling moieties include fluorescent, luminescent, or radionuclide labels. For example, fluorescein isothiocyanate (FITC) may be employed as a fluorescent label in order to provide a quantitative analysis of binding properties. Other suitable labelling moieties include Alexa Fluor dyes, cyanine dyes, and quantum dots. In addition, biotin may be employed as a label for detection means.

The peptide of the invention can be used to retain a wide variety of therapeutic agents in the vitreous. Such agents include antibodies (e.g. bevacizumab), FAB antibody fragments (e.g. ranibizumab), fusion proteins (e.g. aflibercept), peptides (e.g. kinestatin), aptamers (e.g. pegaptanib), and small molecule therapeutics.

In particular, the therapeutic agent may be selected from the group consisting of VEGF inhibitors, alpha2-adrenergic agonists, beta-adrenergic antagonists, Angiotensin II antagonists, ACE inhibitors, NSAIDs, antimalarials, corticosteroids, immune suppressants, monoclonal antibodies, retinoids, DMARDs, biologics, nitrates, prostaglandins, and endothelin antagonists.

Suitable VEGF inhibitors include monoclonal antibodies such as bevacizumab (Avastin), antibody derivatives such as ranibizumab (Lucentis), or molecules that inhibit the tyrosine kinases stimulated by VEGF, such as lapatinib (Tykerb), sunitinib (Sutent), sorafenib (Nexavar), axitinib, and pazopanib. Some of these therapies target VEGF receptors rather than the VEGFs. Tetrahydrocannabinol (THC) and cannabidiol both inhibit VEGF and slow Glioma growth.

Suitable alpha2-adrenergic agonists include apraclonidine, brimonidine, clonidine, detomidine, dexmedetomidine, guanabenz, guanfacine, lofexidine, medetomidine, romifidine, tizanidine, tolonidine, xylazine, fadolmidine, xylometazoline, and oxymetazoline (partial a2 agonist).

Suitable beta-adrenergic antagonists include alprenolol, bucindolol, carteolol, carvedilol, labetalol, nadolol, oxprenolol, penbutolol, pindolol, propranolol, sotalol, timolol, eucommia, acebutolol, atenolol, betaxolol, bisoprolol, celiprolol, esmolol, metoprolol, nebivolol, butaxamine, ICI-118,551, and SR 59230A.

Suitable Angiotensin II antagonists include losartan, irbesartan, olmesartan, candesartan, valsartan and telmisartan.

Suitable ACE inhibitors include Captopril (Capoten), Zofenopril, Enalapril (Vasotec/Renitec), Ramipril (Altace/Prilace/Ramace/Ramiwin/Triatec/Tritace), Quinapril (Accupril), Perindopril (Coversyl/Aceon), Lisinopril (Listril/Lopril/Novatec/Prinivil/Zestril), Benazepril (Lotensin), Imidapril (Tanatril), Zofenopril (Zofecard), Trandolapril (Mavik/Odrik/Gopten), and Fosinopril (Fositen/Monopril).

Suitable NSAIDs include aspirin (acetylsalicylic acid), diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, nimesulide, licofelone, lysine clonixinate, hyperforin, and figwort.

Suitable antimalarials include quinine, chloroquine, amodiaquine, pyrimethamine, proguanil, sulfonamides, mefloquine, atovaquone, primaquine, artemisinin (and derivatives), halofantrine, doxycycline, and clindamycin.

Suitable corticosteroids include hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-butyrate, 17-aceponate, 17-buteprate, and prednicarbate.

Suitable immune suppressants include glucocorticoids, such as hydrocortisone, cortisone, prednisone, prednisolone, methylprednisolone, dexamethasone, betamethasone, triamcinolone, beclometasone, fludrocortisone acetate, deoxycorticosterone acetate (DOCA), and aldosterone, cytostatics, such as nitrogen mustards (cyclophosphamide), nitrosoureas, platinum compounds, and others, folic acid analogues (methotrexate), purine analogues (azathioprine and mercaptopurine), pyrimidine analogues, cytotoxic antibiotics, such as dactinomycin, anthracyclines, mitomycin C, bleomycin, and mithramycin, calcineurin inhibitors (CNIs), such as tacrolimus, and ciclosporin, macrolide lactones, such as sirolimus (rapamycin, trade name Rapamune), interferons, such as IFN-β, opioids, such as codeine, morphine, thebaine, oripavine, diacetylmorphine, nicomorphine, dipropanoylmorphine, diacetyldihydromorphine, acetylpropionylmorphine, desomorphine, methyldesorphine, dibenzoylmorphine, dihydrocodeine, ethylmorphine, heterocodeine, buprenorphine, etorphine, hydrocodone, hydromorphone, oxycodone, oxymorphone, fentanyl, alphamethylfentanyl, alfentanil, sufentanil, remifentanil, carfentanyl, ohmefentanyl, pethidine (meperidine), ketobemidone, allylprodine, prodine, propoxyphene, dextropropoxyphene, dextromoramide, bezitramide, piritramide, methadone, dipipanone, levomethadyl acetate (LAAM), difenoxin, diphenoxylate, loperamide, dezocine, pentazocine, phenazocine, buprenorphine, dihydroetorphine, etorphine, butorphanol, nalbuphine, levorphanol, levomethorphan, lefetamine, meptazinol, tilidine, tramadol, tapentadol, nalmefene, naloxone, and naltrexone, TNF binding proteins, such as infliximab (Remicade), etanercept (Enbrel), adalimumab, curcumin (an ingredient in turmeric), and catechins (in green tea), inosine-5′-monophosphate dehydrogenase (IMPDH) inhibitors, such as mycophenolic acid, and other small biological agents, such as fingolimod, and myriocin.

Suitable monoclonal antibodies include bevacizumab, cetuximab, panitumumab, trastuzumab, infliximab, adalimumab, basiliximab, daclizumab, and omalizumab.

Suitable retinoids include retinol, retinal, tretinoin (retinoic acid, Retin-A), isotretinoin, alitretinoin, etretinate and its metabolite acitretin, tazarotene, bexarotene, and adapalene.

Suitable disease-modifying anti-rheumatic drugs (DMARDs) include adalimumab, azathioprine, ciclosporin, chloroquine and hydroxychloroquine, D-penicillamine, etanercept, golimumab, gold salts (sodium aurothiomalate, auranofin), infliximab, leflunomide, methotrexate (MTX), minocycline, rituximab, and sulfasalazine (SSZ).

Suitable biologics include abciximab, etanercept (Enbrel), infliximab (Remicade), rituximab (Rituxan), trastuzumab (Herceptin), and ocriplasmin (Jetrea).

Suitable nitrates include glyceryl trinitrate (GTN), isosorbide dinitrate, and isosorbide mononitrate.

Suitable prostaglandins include prostacyclin I₂ (PGI₂), prostaglandin E₂ (PGE₂), and prostaglandin F_{2α} (PGF_{2α}).

Suitable endothelin antagonists include sitaxentan, ambrisentan, atrasentan, BQ-123, zibotentan, bosentan, macitentan, tezosentan, BQ-788, and A192621).

In particular, the binding conjugates of the present invention may be employed in conjunction with VEGF inhibitors. This arrangement has a number of benefits over other potential treatments for certain conditions. For example, unlike potential gene therapy methods for the long term intravitreal delivery of VEGF inhibitors, the side effect profile of simple biological agents is relatively well understood (worldwide experience with ranibizumab, pegaptanib, bevacizumab, and aflibercept). As a result, delivering such biological agents using the technology of the invention means that the biological profile of the administered agent can be accurately predicted and managed. Furthermore, in instances where adverse reactions are found to occur, a vitrectomy could be performed to remove the formulation of the invention, but similar abrogation of treatment is currently not available using gene therapy.

The diagnostic agent of the collagen or hyaluronic acid binding conjugate may comprise a fluorescent, luminescent, or radionuclide label. For example, specific diagnostic agents include sodium fluorescein and indocyanine green.

In another embodiment of the invention, there is provided a pharmaceutical composition comprising a peptide according to the invention, or a collagen or hyaluronic acid binding conjugate according to the invention, and at least one pharmaceutically acceptable excipient.

In particular, it has been found that due to the ability of the binding moiety to target specific tissues of the human or animal body, the peptide according to the invention, the collagen or hyaluronic acid binding conjugate according to the invention, or the pharmaceutical composition according to the invention, is suitable for use in therapy, specifically the prophylaxis or treatment of age-related macular degeneration, diabetic retinopathy, diabetic macular oedema, retinal vein occlusion, retinopathy of prematurity, pathologic myopia macular oedema, macular telangiectasia, choroidal neovascularisation, uveitis, glaucoma, systemic lupus erythematosus, arthritis, rheumatoid arthritis, scleroderma, polymyositis, or dermatomyositis. Preferably, ocular diseases or conditions, such as age-related macular degeneration, diabetic retinopathy, diabetic macular oedema, retinal vein occlusion, retinopathy of prematurity, pathologic myopia macular oedema, macular telangiectasia, choroidal neovascularisation, uveitis, or glaucoma are applicable. In particular, age-related macular degeneration, diabetic retinopathy, retinal vein occlusion, uveitis, and glaucoma are preferred medical indications which may be prevented or treated with the peptide, conjugate or pharmaceutical composition according to the invention.

The pharmaceutical composition may comprise an excipient which enables the binding conjugate to be delivered to the relevant site for use. The excipient may target a particular site or otherwise improve delivery to that site. It may also comprise an excipient which stabilises the binding conjugate. Any appropriate stabiliser may be used.

Pharmaceutical compositions of the invention may comprise any pharmaceutically acceptable carrier, adjuvant or vehicle. Pharmaceutically acceptable carriers, adjuvants and vehicles that may be used in the pharmaceutical compositions include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention may be administered orally, parenterally, by inhalation spray, topically, rectally, nasally, buccally, vaginally or via an implanted reservoir. Preferably, the pharmaceutical compositions are administered topically, via an implanted reservoir or by injection (more preferably by injection). The pharmaceutical compositions may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrasternal, intrathecal, intraocular, intralesional and intracranial injection or infusion techniques. Preferably, the route of administration of the composition is intraocular or intra-articular administration.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

Topical administration of the pharmaceutical compositions of the invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the molecules of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

In particular, the binding conjugates of the present invention can be formulated into a clear solution. This therefore ensures that visual clouding does not occur when the moieties are employed to treat ocular conditions, and alleviates any payload issues associated with existing treatments.

Where the binding conjugate comprises a peptide according to the invention, it has also been advantageously found that the formulation has inherent antibacterial/anti-inflammatory properties. This further minimises the potential for acquired infection when the formulation is parentally administered.

In one embodiment, it may also be desirable for the pharmaceutical composition to include at least one additional unconjugated therapeutic agent, i.e. which is not covalently linked to the collagen or hyaluronic acid binding conjugate. In this case, formulations including a mixture of therapeutic agents, both conjugated and unconjugated, allow for both short acting (i.e. the unconjugated agents) and long acting components (i.e. the conjugated agents) to be present in the same formulation.

Suitable additional unconjugated therapeutic agents include those selected from the group consisting of VEGF inhibitors, alpha2-adrenergic agonists, beta-adrenergic antagonists, Angiotensin II antagonists, ACE inhibitors, NSAIDs, antimalarials, corticosteroids, immune suppressants, monoclonal antibodies, retinoids, DMARDs, biologics, nitrates, prostaglandins, and endothelin antagonists.

In a further embodiment of the invention, there is provided a use of an isolated peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, and wherein the N-terminus of the peptide comprises a D-amino acid and/or includes a protecting group, for preparing a collagen or hyaluronic acid binding conjugate.

Preferably, the use relates to a peptide having a sequence with at least 60% homology to SEQ ID No. 1, or a functional portion or fragment thereof, for preparing a collagen or hyaluronic acid binding conjugate. More preferably, the functional portion or fragment comprises at least 5 contiguous amino acids from SEQ ID No. 1 and shows at least 70% of the affinity of the peptide having at least 60% homology to SEQ ID No. 1 to hyaluronic acid and/or at least 70% of the affinity of the peptide having at least 60% homology to SEQ ID No. 1 to collagen. In addition or alternatively, the peptide is a functional portion or fragment thereof having a sequence according to any of those shown in Table 1, and which shows at least 70% of the affinity of the peptide having at least 60% homology to SEQ ID No. 1 to hyaluronic acid and/or at least 70% of the affinity of the peptide having at least 60% homology to SEQ ID No. 1 to collagen.

In another embodiment of the invention, there is provided an isolated peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, for use in the prophylaxis or treatment of ocular diseases or conditions, such as age-related macular degeneration, diabetic retinopathy, diabetic macular oedema, retinal vein occlusion, retinopathy of prematurity, pathologic myopia macular oedema, macular telangiectasia, choroidal neovascularisation, uveitis, or glaucoma. Preferably, the peptide for use in this manner is an isolated peptide having a sequence with at least 60% homology to SEQ ID No. 1, or a functional portion or fragment thereof. It is also preferable that the N-terminus of the peptide comprises a D-amino acid and/or includes a protecting group.

There is also provided a collagen or hyaluronic acid binding conjugate comprising a peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, and a therapeutic or diagnostic agent, wherein the therapeutic or diagnostic agent is optionally bound to the peptide by means of a linker, for use in the prophylaxis or treatment of ocular diseases or conditions, such as age-related macular degeneration, diabetic retinopathy, diabetic macular oedema, retinal vein occlusion, retinopathy of prematurity, pathologic myopia macular oedema, macular telangiectasia, choroidal neovascularisation, uveitis, or glaucoma.

In yet a further embodiment of the invention, there is provided a method of detecting a hyaluronic acid binding substance, the method comprising providing a sample of hyaluronic acid, contacting the sample of hyaluronic acid with a test substance, and detecting the presence of binding between the test substance and the hyaluronic acid. In particular, the hyaluronic acid may be non-covalently bound to a solid support. If this is the case, the solid support is preferably an amine surface.

In addition or alternatively, the method preferably utilises bovine serum albumin as a blocking agent and/or as a diluent.

The means of detection is not particularly limited and may involve any common detection method which is used for similar enzyme-linked immunosorbent assays (ELISAs). Preferably, however, the detection method is carried out using a biotinylated substrate (e.g. a biotinylated recombinant protein) and streptavidin-horse radish peroxidase, with addition of a peroxidase substrate, such as tetramethylbenzidine chloride. Biotinylated recombinant human aggrecan may be used as a positive control.

In another embodiment of the invention, there is provided a method of preventing or treating a condition associated with age-related macular degeneration, diabetic retinopathy, diabetic macular oedema, retinal vein occlusion, retinopathy of prematurity, pathologic myopia macular oedema, macular telangiectasia, choroidal neovascularisation, uveitis, glaucoma, systemic lupus erythematosus, arthritis, rheumatoid arthritis, scleroderma, polymyositis, or dermatomyositis, comprising administering to a subject in need thereof the peptide according to the invention (i.e. a peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, wherein the N-terminus of the peptide comprises a D-amino acid and/or includes a protecting group), the collagen or hyaluronic acid binding conjugate according to the invention, or the pharmaceutical composition according to the invention.

There is also provided a method of preventing or treating an ocular disease or condition, such as age-related macular degeneration, diabetic retinopathy, diabetic macular oedema, retinal vein occlusion, retinopathy of prematurity, pathologic myopia macular oedema, macular telangiectasia, choroidal neovascularisation, uveitis, or glaucoma, comprising administering to a subject in need thereof a peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids.

In a further embodiment, there is provided a method of preventing or treating an ocular disease or condition, such as age-related macular degeneration, diabetic retinopathy, diabetic macular oedema, retinal vein occlusion, retinopathy of prematurity, pathologic myopia macular oedema, macular telangiectasia, choroidal neovascularisation, uveitis, or glaucoma, comprising administering to a subject in need thereof a collagen or hyaluronic acid binding conjugate comprising a peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, and a therapeutic or diagnostic agent, wherein the therapeutic or diagnostic agent is optionally bound to the peptide by means of a linker.

The invention will now be described in more detail by way of example only and with reference to the following figures.

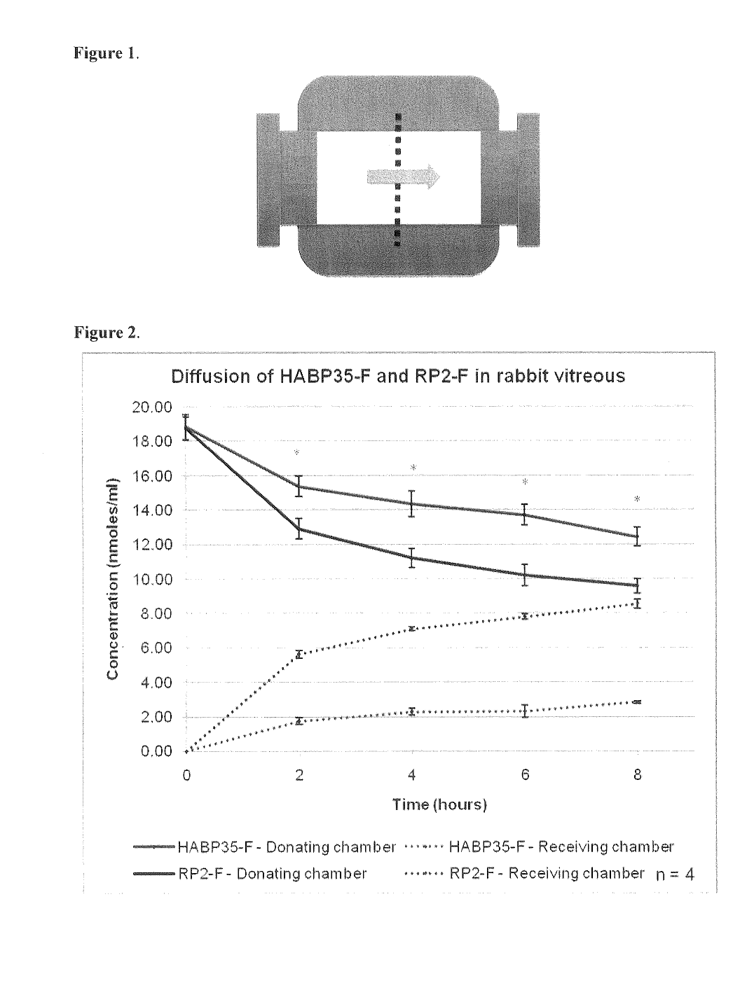

FIG. 1. Harvard Fast Micro-Equilibrium Dialyzer. Each dialyzer is made of polytetrafluoroethylene (PTFE) and contains a 250 μl chamber on either side of a 100 kDa molecular weight cut off (MWCO) cellulose acetate membrane. The chambers were incubated at room temperature for up to 8 hours, and the diffusion from one chamber to the other was assessed. The side in which the peptide is placed is termed the donating chamber. The other side, towards which the peptide diffuses, is termed the receiving chamber.

FIG. 2. The diffusion of HABP35-F (outer lines) and RP2-F (inner lines) in rabbit vitreous, using a Harvard Fast Micro-Equilibrium Dialyzer. At the start of the experiment the donating chamber contained 200 μl of rabbit vitreous with 20 mole/ml of either HABP35-F or RP2-F. The receiving chamber contained 200 μl of rabbit vitreous without peptide. Peptide diffusion across the 100 kDa MWCO cellulose acetate filter was measured over time. At each time point the vitreous from each chamber was removed and peptide concentration quantified by measuring fluorescence. Four chambers were prepared for each time point (n=4). HABP35-F concentration, in the donating chamber, is significantly higher than RP2-F, at 2, 4, 6 and 8 hours (*p<0.05—One-way ANOVA, Bonferroni post test). HABP35-F was therefore retained, in the donating chamber, to a significantly greater extent than the control peptide RP2-F. Error bars represent standard deviation.

FIG. 3. The diffusion of HABP35-F (lower line) and RP2-F (higher line) in rabbit vitreous, using a Harvard Fast Micro-Equilibrium Dialyzer. This graph shows the concentration gradient (concentration difference) between the donating and receiving chambers at each time point. Concentration gradient=concentration in donating chamber−concentration in receiving chamber. At 2, 4, 6 and 8 hours the concentration gradient for HABP35-F is significantly higher then for the control peptide RP2-F (*p<0.05—One-way ANOVA, Bonferroni post test). HABP35-F was therefore retained, in the donating chamber, to a significantly greater extent than the control peptide RP2-F. Error bars represent standard deviation.

FIG. 4. The diffusion of HABP35-F (left-hand column in each case) and RP2-F (right-hand column in each case) in hyaluronic acid, using a Harvard Fast Micro-Equilibrium Dialyzer. Instead of rabbit vitreous, each chamber was loaded with either 2.5 mg/ml hyaluronic acid (HA), or HEPES buffered saline (HBS). Concentration of HABP35-F or RP2-F, in the donating and receiving chambers, was measured at the 8-hour time point (n=3). The concentration gradient was then determined (difference between the concentration of the donating and receiving chambers). In the presence of HA, HABP35-F was retained in the donating chamber, to a significantly greater extent than the control peptide RP2-F. (*p<0.05—One-way ANOVA, Bonferroni post test) as shown by a significantly greater concentration gradient. This was not the case in the presence of HBS alone. The retention of HABP35-F in rabbit vitreous is therefore due to an interaction with HA. Error bars represent standard deviation.

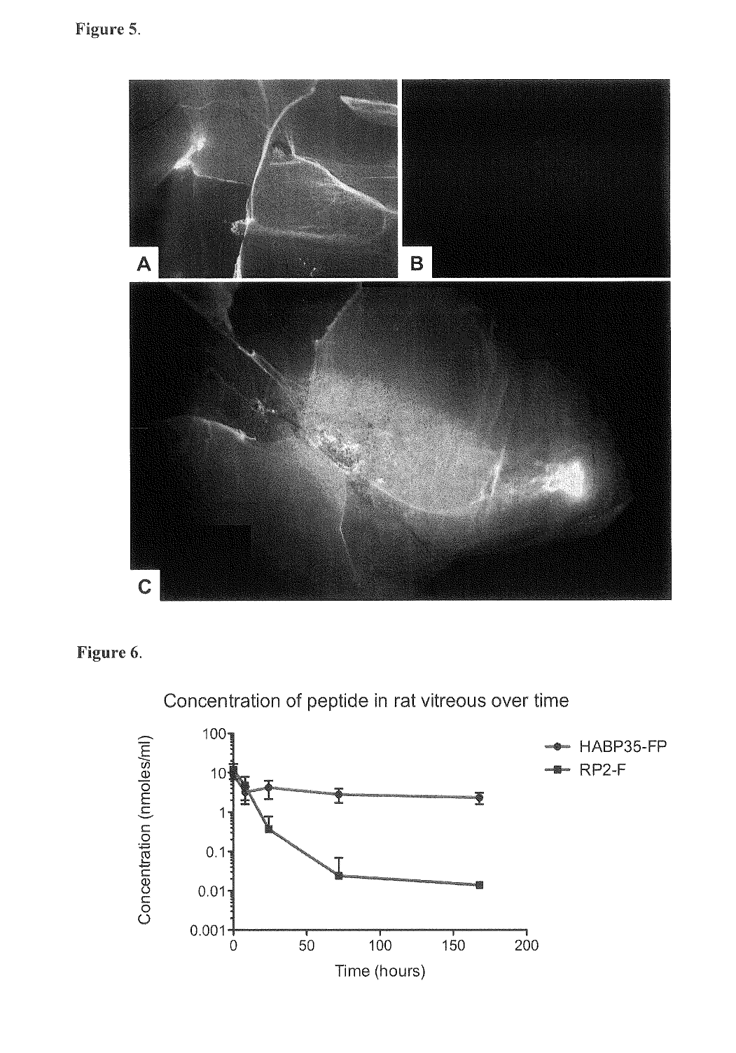

FIG. 5. The retention of HABP35-F and RP2-F in rat vitreous after 48 hours. 2.5 μl of HABP35-F or RP2-F (250 nmole/ml) was injected into the vitreous of adult male Sprague Dawley rats. After 48 hours the eyes were removed. The cornea and lens was removed and the eye cup opened out into a Maltese cross. The vitreous and retina was photographed using an epifluorescent microscope (using equal exposure settings). A) Fluorescence of HABP35-F in the vitreous 48 hours following injection. B) Fluorescence of RP-F in the vitreous 48 hours following injection. C) Collage photograph showing fluorescence of HABP35-F in the vitreous 48 hours following injection. After 48 hours there is visibly more fluorescent peptide seen in the vitreous following injection of HABP35-F as opposed to control peptide RP2-F.

FIG. 6. The in vivo diffusion study of HABP35-FP (higher line) and RP2-F (lower line) in rat vitreous over time. 2.5 μl of 250 nmole/ml peptide in HBS was injected into the vitreous of adult male Sprague Dawley rats. At different time points (0, 8, 24, 72, 168 hours) rats were sacrificed and the concentration of peptide in the vitreous was measured by fluorescence. Three eyes were used at each time point, for each peptide (n=3). There was increased retention of HABP35-FP in the rat vitreous, over time, as compared with RP2-F. The difference in concentrations between the two peptides was statistically significant at 24, 72 and 168 hours (p=0.034, 0.011 and 0.006 respectively—unpaired t test). Error bars represent standard deviation.

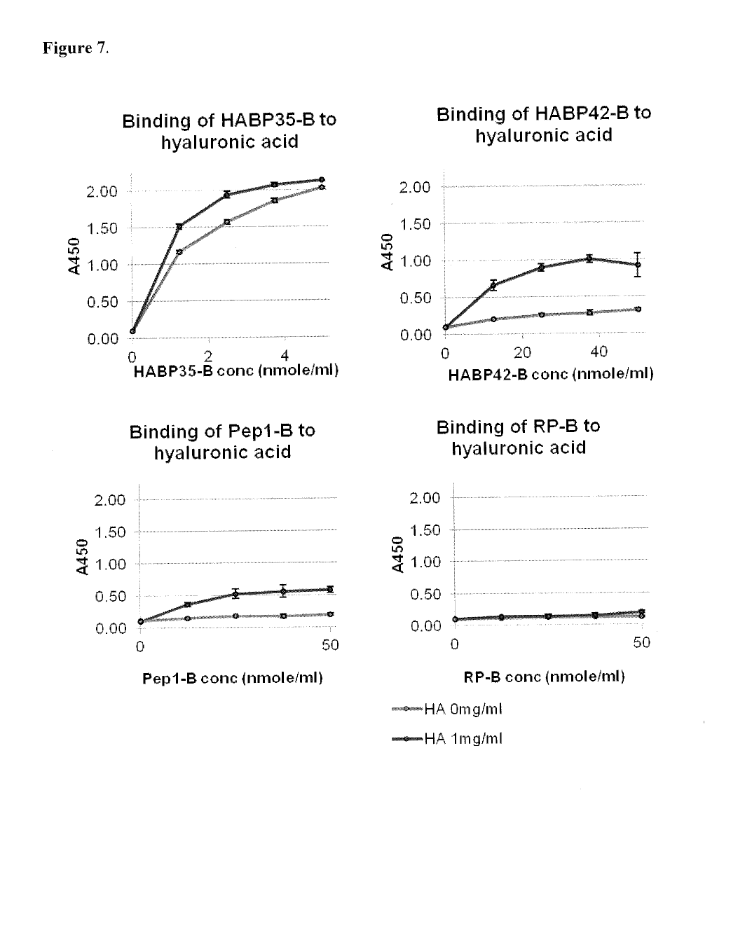

FIG. 7. The binding of Pep1-B, HABP42-B, HABP35-B and RP-B to non-covalently bound HA. The four graphs show binding to blocked wells in the presence (higher line in each case) and absence (lower line in each case) of HA (total binding and non-specific binding respectively). Three ELISAs were performed for each concentration (n=3). Error bars represent standard deviation.

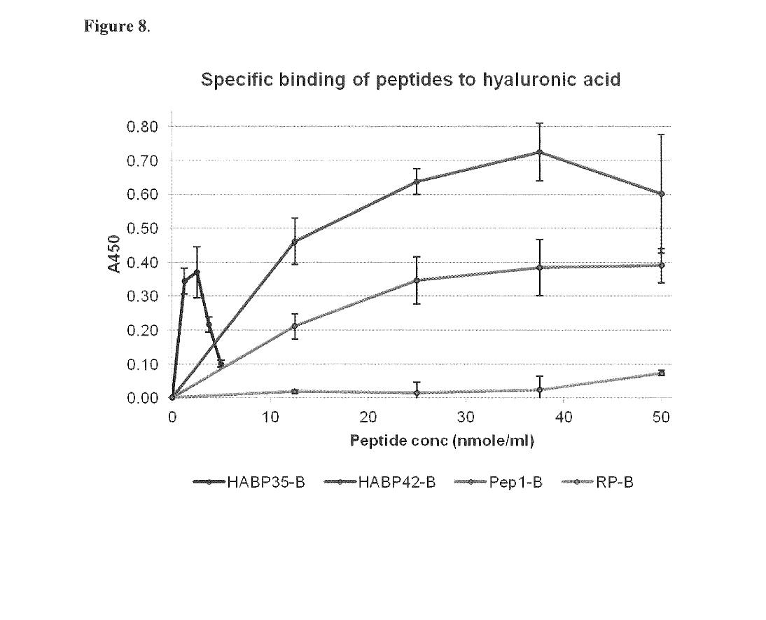

FIG. 8. The specific binding of HABP35-B, HABP42-B, Pep1-B, and RP-B (in order of greatest initial response at the origin) to non-covalently bound HA. Specific binding was obtained by subtracting A450 of blank wells from A450 of HA loaded wells, from the readings represented in FIG. 7. Three ELISAs were performed for each concentration (n=3). Both HABP42-B and Pep1-B showed specific binding to hyaluronic acid, which reached saturation (as seen by plateau formation) at a concentration of between 25 and 50 nmole/ml. When compared with RP-B, both HABP42-B and Pep1-B showed significantly greater specific binding at all concentrations tested (p<0.0001—One-way ANOVA). HABP35-B showed specific binding at concentrations ten-fold less than HABP42-B or Pep1-B. However due to high non-specific binding, its specific signal deteriorated at higher concentrations. Error bars represent standard deviation.

FIG. 9. The chemistry of crosslinking. A) Anti-IL-1β antibody (mouse anti-human) (left) was covalently linked to the hyaluronic acid binding peptide (HABP35) (right) using a crosslinker (EMCH) (middle). The reactive groups are indicated by three letter abbreviations: Ald=Aldehyde, Hyd, =Hydrazide, Mal, =Maleimide, Sul=Sulphydryl. Reaction 1 took place before reaction 2. B) Reaction 1—HABP35 was manufactured with a cysteine residue at the C-terminus. This allowed crosslinking between the sulphydryl group on the cysteine residue and the maleimide reactive group on the cross-linker, forming a stable thioether bond. C) Reaction 2—Sugar residues on the antibody were oxidized to form aldehyde groups. These were then cross-linked with the hydrazide reactive group on the crosslinker, forming a stable hydrazone bond. (Figure parts B & C courtesy of Piercenet Ltd). Ketone or aldehyde groups can be created in glycoproteins by oxidizing the polysaccharide post-translational modifications (glycosylation) with sodium meta-periodate. As carbonyl groups are present on the sugar residues of antibodies, cross-linking to these groups has the advantage of not altering the antigen-binding site of the antibody.

FIG. 10. The effect of bestatin on degradation of HABP35-F by rabbit vitreous. Bestatin (250 μM) significantly increased the amount of HABP35-F (10 nmole/ml) detectable by UPLC-MS after 6 hours incubation in rabbit vitreous (p=0.01). UPLC-MS=ultra performance liquid chromatography-mass spectrometry.

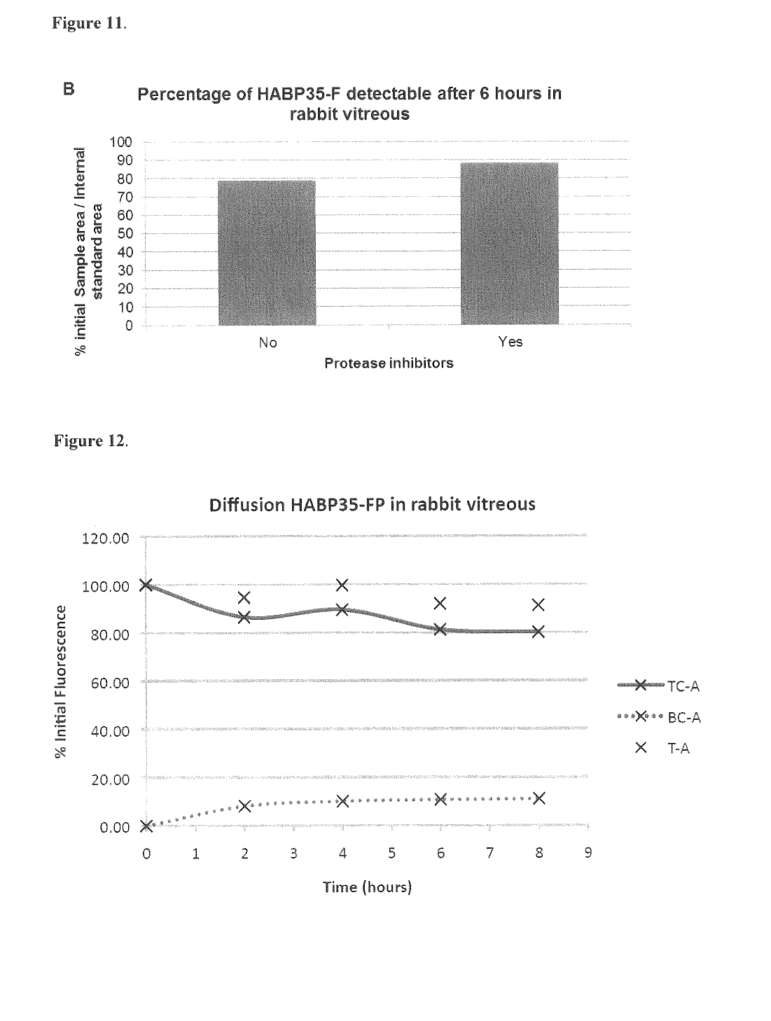

FIG. 11. The effect of the protease inhibitors aprotinin (8 μM), bestatin (500 μM), E-64 (150 μM), and leupeptin (200 μM) on HABP35-F detection by UPLC-MS, after 6 hours incubation in rabbit vitreous. The protease inhibitor cocktail appeared to increase the amount of HABP35-F detected. The experiment was performed three times (n=3). UPLC-MS=ultra performance liquid chromatography-mass spectrometry.

FIG. 12. The diffusion of HABP35-FP in rabbit vitreous, using a Harvard Fast Micro-Equilibrium Dialyzer. At each time point, the vitreous from each chamber was removed and peptide concentration quantified by measuring fluorescence. HABP35-FP was retained in the donating chamber to a significantly extent, and the total amount of peptide (in either chamber) indicated that minimal degradation of the peptide was observed. TC-A corresponds to the donating chamber; BC-A corresponds to the receiving chamber; T-A corresponds to the total amount of peptide.

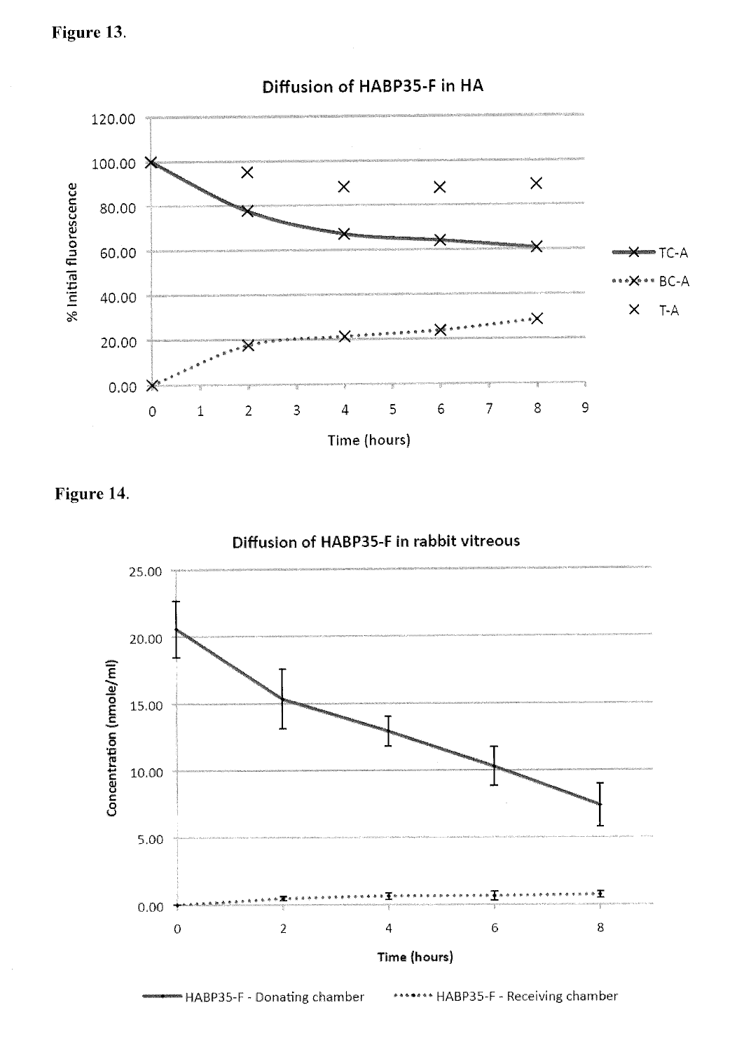

FIG. 13. The diffusion of HABP35-F in hyaluronic acid, using a Harvard Fast Micro-Equilibrium Dialyzer. At each time point, the hyaluronic acid from each chamber was removed and peptide concentration quantified by measuring fluorescence. HABP35-F was retained in the donating chamber to a significantly extent, and the total amount of peptide (in either chamber) indicated that minimal degradation of the peptide was observed. TC-A corresponds to the donating chamber; BC-A corresponds to the receiving chamber; T-A corresponds to the total amount of peptide.

FIG. 14. The diffusion of HABP35-F in rabbit vitreous, using a Harvard Fast Micro-Equilibrium Dialyzer. At each time point, the vitreous from each chamber was removed and peptide concentration measured. HABP35-F was retained in the donating chamber, although the inconsistency between the concentrations of HABP35-F in the donating and receiving chambers suggest that some degradation of the peptide occurred.

### EXAMPLES

### Example 1

### Diffusion Properties of HABP35-F in the Vitreous

Labelled HABP35 was manufactured (GenScript Inc, USA) with a linker sequence (GGGS) added to the C-terminal region to prevent steric hindrance between the labelling molecule and HABP35 occurring. The C-terminal lysine residue was labelled with fluorescein isothiocyanate (FITC). The sequence of the modified HABP35 peptide (HABP35-F) is a follows:

HABP35-F
LKQKIKHVVKLKVVVKLRSQLVKRKQN-GGGS-K(FITC)-amide

### Purity: 95.3%

Molecular weight: 4013.9

A control peptide (RP2-F) was also manufactured with a molecular weight similar to HABP35. It was designed using a sequence already shown to show no significant binding to hyaluronic acid (Mummert et al. Development of a peptide inhibitor of hyaluronan-mediated leukocyte trafficking. J Exp Med. 2000; 18; 192(6): 769-779):

RP2-F
SATPASAPYPLAGGGSSATPASAPYPLAGGGS-K(FITC)-amide

### Purity: 95.1%

Molecular weight: 3305.61

The purity of both peptides was confirmed using high performance liquid chromatography. The molecular mass was confirmed using electrospray mass spectrometry. The peptides were renamed HABP35-F and RP2-F, respectively.

In order to study the diffusion properties of HABP35-F in vitreous, Fast Micro-Equilibrium Dialyzers (250 μl chamber volume) were purchased from Harvard Apparatus Ltd (UK). Each dialyzer contains two 250 μl chambers separated by a cellulose acetate membrane (molecular weight cut off of 100 kDa) (FIG. 1). 200 μl of rabbit vitreous (Pel-freez Ltd, USA) containing 20 nmole/ml HABP35-F or RP2-F was placed in one side of the dialyser (donating chamber), while 200 μl of rabbit vitreous (no peptide) was placed in the other side (receiving chamber). The rate at which HABP35-F and RP2-F diffused from one side of the dialyzer to the other was assessed over time. Four dialyzers were set up for each diffusion run, to allow sampling at 2, 4, 6, and 8 hours (one dialyzer per time point).

At each time point vitreous from each side of one dialyzer was sampled. The concentration of peptide in each chamber was quantified by measuring fluorescence (excitation wavelength 490 nm, emission wavelength 510-570 nm (corresponding to the peak fluorescence of fluorescein). Fluorescence was compared to a standard concentration curve for each peptide, in order to obtain concentration values.

Over the eight hour period the control peptide RP2-F diffused across the membrane, almost reaching equilibrium (concentration in the donating chamber equal to the concentration in the receiving chamber) (FIG. 2). The concentration gradient (difference in concentration between donating and receiving chamber) almost reached zero (FIG. 3). HABP35-F diffused more slowly across the membrane, and by eight hours there was a significantly greater concentration left in the donating chamber (as compared to RP2-F) (FIG. 2). The concentration gradient also remained significantly higher for HABP35-F indicating retention in the donating chamber (FIG. 3).

Young rabbit vitreous humour was used in the experiment described above (Pel-freez Biologicals Ltd). It was initially defrosted, aliquoted, and refrozen at −20° C. Defrosted samples were then brought to a physiological pH of 7.2-7.4 through the addition of 1.8% HCl. The rabbit vitreous was then centrifuged at 13 000 for 10 minutes, to remove any insoluble material, in a Heraeus Biofuge Fresco centrifuge (Kendro Laboratory Products Ltd).

### Example 2

### Binding Properties of HABP35-F to Hyaluronic Acid

In order to assess whether the retention in the donating chamber was due to an interaction with hyaluronic acid (HA), a solution of 2.5 mg/ml HA (in HEPES buffered saline) was added to each chamber, instead of rabbit vitreous. 20 nmole/ml of HABP35-F or RP2-F was added to the donating chamber and diffusion was measured over 8 hours. Three chambers were used for each time point (n=3). As a control, diffusion in HEPES buffered saline (HBS) alone was also assessed. After 8 hours there was a significantly greater concentration gradient for HABP35-F, as compared to RP2-F, in HA. This was not the case when peptide diffusion was assessed in HBS alone (FIG. 4). This indicates that HABP35-F retention in rabbit vitreous may be, at least in part, due to an interaction with HA.

### Example 3

### Degradation Studies

In order to assess degradation of HABP35-F by proteases present in rabbit vitreous, HABP35-F was incubated in rabbit vitreous for a period of 12 hours. Samples of HABP35-F in rabbit vitreous were taken at 0 and 12 hours and the mass spectrum traces for HABP35-F were compared. At 0 hours, the mass spectrum contained ions representing intact HABP35-F. At 12 hours, these ions were still present (m/z values of 502.3, 574.1, 669.5, and 803.7, representing a molecule with a MW of 4012). However, a new set of ions were also detected at 12 hours (m/z values of 557.9, 651.0 and 781.0, representing a new molecule with a MW of 3899). This new molecule was 113 Da lighter than intact HABP35-F. Loss of the N-terminal leucine residue, via peptide hydrolysis, would lead to a peptide fragment 113 Da lighter than HABP35-F. It was therefore believed that this new molecule was HABP35-F minus the N-terminal leucine.

This new molecule did not appear in the absence of HABP35-F, indicating that it originated from this peptide. In order to confirm that fragmentation occurred at the N-terminus (as opposed to the C-terminus), HABP35-B was incubated for 12 hours with rabbit vitreous. With HABP35-B a new ion also appeared, containing a comparable loss in MW to that seen with HABP35-F. This indicated that the both HABP35-F and HABP35-B undergo alteration at their identical N-termini as opposed to their different C-termini (different due to different labelling modifications).

In order to further confirm that HABP35-F was being enzymatically digested at the N-terminus, the proportion of HABP35-F that could be detected following incubation was assessed, with or without bestatin. Bestatin is an aminopeptidase inhibitor. Aminopeptidases catalyse the cleavage of amino acids from the N-terminus of peptides/proteins. Bestatin significantly increased the proportion of HABP35-F that was detected after 6 hours of incubation with rabbit vitreous (p=0.01—unpaired student t-test) (FIG. 10). This confirmed that aminopeptidases were involved in enzymatic digestion of HABP35-F.

Although this degradation does not render the peptides ineffective for clinical treatments, the vulnerability of the N-terminal leucine residue to enzymatic digestion in rabbit vitreous was problematic as it reduced the ability to quantify all the remaining peptide (both the intact and fragmented version) using mass spectrometry in SIM mode. The HABP35-F fragment would not be detected when scanning for HABP35-F using SIM mode. In order to optimise detection, further other protease inhibitors were added (aprotinin, bestatin, E-64, leupeptin). These protease inhibitors showed a trend towards protection of HABP35-F (FIG. 11).

This degradation was not seen when the N-terminus of the peptide comprised a D-amino acid and/or included a protecting group. For example, the level of HABP35-FP in a diffusion study in rabbit vitreous was observed to be almost constant between the donating and receiving chambers, and retention in the donating chamber was clearly evident (FIG. 12). Similar results were observed for the diffusion of HABP35-F in hyaluronic acid (FIG. 13), but the diffusion of HABP35-F in rabbit vitreous showed a significant loss in the total amount of peptide (FIG. 14).

### Example 4

### In Vivo Model of HABP35 Retention

HABP-35-F was modified to protect the N-terminus from enzymatic degradation, through conversion of the terminal leucine to its D-configuration and by acetylation. The new peptide was called HABP35-FP. Adult male Sprague Dawley rats were used as the in vivo model. 2.5 μl of 250 nmole/ml HABP35-FP or RP2-F was injected into the vitreous and animals were culled at various time points. The vitreous was extracted and peptide concentration measured by assessing fluorescence of the extracted vitreous. Three eyes were assessed, for each peptide, at each time point. In addition, the fluorescence of the vitreous was directly assessed on eye-cup flat mounts using an epifluorescent microscope.

There was increased retention of HABP35-FP, as compared to RP2-F, on epifluorescent microscopy, at 48 hours (FIG. 5). This retention extended to at least 168 hours following injection (FIG. 6).

### Example 5

### Preparation of Peptide-Antibody Complex

Each cysteine labeled peptide (HABP35-C or RP2-C) was dissolved in 1000 μl of degassed phosphate buffered saline (PBS, pH 7.2, Invitrogen Ltd) to a concentration of 250 μM. 50 μl of 50 mM 3,3′-N-[ε-Maleimidocaproic acid] hydrazide, trifluoroacetic acid salt (EMCH), dissolved in dimethyl sulphoxide (DMSO, Sigma-Aldrich Ltd), was immediately added. The mixture was covered in argon and sealed to prevent oxidative formation of disulphide bonds. It was protected from light and incubated at room temperature for 2 hours.

To remove any unlinked EMCH, the reaction mixture was dialysed against 500 ml PBS in a 3 ml 2 kDa molecular weight cut-off (MWCO) Slide-A-Lyzer Dialysis Cassette (Thermo Fisher Scientific/Pierce Ltd). The PBS was changed at 6 and 12 hours, with dialysis completed by 24 hours. Dialysis was performed at 4° C.

250 μg mouse monoclonal anti human IL-113 antibody (R&D Systems Ltd) was dissolved in 500 μl cold sterile PBS. Sodium meta-periodate (Thermo Fisher Scientific/Pierce Ltd) was dissolved in oxidation buffer (20 mM sodium acetate, pH 5.5) to a concentration of 20 mM. A volume was prepared equal to the volume of antibody (500 μl). This solution was kept on ice and protected from light. 500 μl of cold sodium meta-periodate solution was added to 500 μl of antibody solution. It was quickly brought to room temperature and incubated for 30 minutes, protected from light, on a SB3 Variable Speed Rotary Mixer at 20 rpm (Stuart Ltd). Buffer exchange (oxidation buffer replaced with PBS) was performed using 5 ml 7 kDa MWCO Zeba Spin Desalting Columns (Thermo Fisher Scientific/Pierce Ltd) according to the product protocol.

The peptide-EMCH complex was mixed with the oxidised antibody. The mixture was incubated at room temperature for 2 hours on an orbital shaker (Heidolph Ltd) at 30 rpm. To remove any unlinked peptide-EMCH complex, the reaction mixture was dialysed against 500 ml PBS in a 3 ml 20 kDa MWCO Slide-A-Lyzer Dialysis Cassette. The PBS was changed at 6 and 12 hours, with dialysis completed by 24 hours. Dialysis was performed at 4° C.

The peptide-EMCH-antibody complex was then filter sterilized using a Costar Spin-X 0.22 μm cellulose acetate centrifuge tube filters (Corning Ltd). It was then concentrated using Amicon Ultra 30 kDa MWCO centrifugal filter units (Millipore Ltd).

### Example 6

### HA Binder Screening Method

Wells of a clear polystyrene Amine Surface 96 well ELISA plates (Corning Life Sciences Ltd) were loaded with 100 μl of 1 mg/ml HA sodium salt (Sigma-Aldrich Ltd) in 0.1 M 2-[N-morpholino] ethane sulfonic acid (MES, pH 4.5-5, Sigma-Aldrich Ltd). The wells were incubated for three hours at room temperature on an orbital shaker. All further incubations occurred at room temperature on an orbital shaker.

Wells were then washed three times with wash buffer (0.05% Tween 20 (Sigma-Aldrich Ltd) in phosphate buffered saline (PBS), pH 7.2-7.4). PBS was prepared to the following formula: 137 mM sodium chloride, 2.7 mM potassium chloride, 8.1 mM sodium phosphate dibasic, 1.5 mM potassium phosphate monobasic, pH 7.2-7.4, 0.22 μm filtered. 300 μl of 3% BSA (Sigma-Aldrich Ltd) in PBS was used to block each well. After 90 minutes incubation the wells were washed again three times. Different concentrations of biotinylated HA binding peptides, or control peptides, were then added, dissolved in 100 μl of 3% BSA PBS, and incubated for 1 hour. Three further washes were performed. 100 μl of streptavidin-horse radish peroxidase (S-HRP) (R&D Systems Ltd) (diluted to a working concentration of 1:200 in PBS) was added to each well, to detect any bound biotinylated peptide. The wells were incubated for 20 minutes, protected from light, followed by three further washes. 100 μl of tetramethylbenzidine (TMB)/H₂O₂, was added to each well. The wells were incubated for 10 minutes, protected from light. The reaction was stopped with 50 μl of 1 M H₂SO₄. Optical density of each well was read immediately, using a Modulus Microplate Reader set to 450 nm, One-way analysis of variance (ANOVA) was used to deter line statistical significance between groups (GraphPad Prism 5, GraphPad software Ltd).

Table 2 summarises the parameters of the method.

TABLE 2

| | | | Duration | Temp. |
|---|---|---|---|---|
| Step | Reagent/Product | Diluent | (min) | (° C.) |
| Plate | Amine Surface plate | NA | NA | NA |
| Sample | 1 mg/ml HA | 0.1M MES | 180 | 21 |
| | | (pH 4.5-5.0) | | |
| Blocking | 3% BSA | PBS | 90 | 21 |
| Detection | HABP (variable | 3% BSA in PBS | 60 | 21 |
| | conc) | | | |
| | 1:200 S-HRP | PBS | 20 | 21 |
| | TMB/H₂O₂ | NA | 10 | 21 |
| | H₂SO₄ | NA | NA | 21 |

Abbreviations: HA, Hyaluronic acid, MES, 2-(N-morpholino)ethanesulfonic acid, PBS, Phosphate buffered saline, S-HRP, Streptavidin-horse radish peroxidise, TMB, Tetramethylbenzidine.

### Example 7

### Peptides and Conjugates

HABP35:
(SEQ ID No. 1)
LKQKIKHVVKLKVVVKLRSQLVKRKQN-amide
HABP35-F:
(SEQ ID No. 239)
LKQKIKHVVKLKVVVKLRSQLVKRKQN-GGGS-K(FITC)-amide
HABP35-FP
(SEQ ID No. 240)
Acetyl D-(L) KQKIKHVVKLKVVVKLRSQLVKRKQN-GGGS-
K(FITC)-amide
HABP35-C
(SEQ ID No. 241)
Acetyl D-(L) KQKIKHVVKLKVVVKLRSQLVKRKQN-GGGS-
K(Biotin)-C-amide
Biotin labelling at the N-terminus:
(SEQ ID No. 242)
Biotin-LKQKIKHVVKLKVVVKLRSQLVKRKQN-amide
Biotin labelling at the C-terminus:
(SEQ ID No. 243)
LKQKIKHVVKLKVVVKLRSQLVKRKQN-GGGS-K(Biotin)-amide
Protection of the N-terminus:
(SEQ ID No. 244)
Acetyl D-(L) KQKIKHVVKLKVVVKLRSQLVKRKQN-GGGS-
K(FITC)-amide
Kinestatin-HABP35
(SEQ ID No. 245)
{pGlu}IPGLGPLR-GGGS-LKQKIKHVVKLKVVVKLRSQLVKRKQN-
amide
HABP35-Kinestatin
(SEQ ID No. 246)
Acetyl-{d-Leu}KQKIKHVVKLKVVVKLRSQLVKRKQN-GGGS-
QIPGLGPLR-amide

### Reference Peptides:

HABP42-F:
(SEQ ID No. 247)
D-(STMMSRSHKTRSHHV)L-(GGGS-K(FITC)-amide)
Pep1-B:
(SEQ ID No. 248)
GAHWQFNALTVR-GGGS-K(Biotin)-amide
RP-F:
(SEQ ID No. 249)
SATPASAPYPLA-GGGS-K(FITC)-amide
RP2-F:
(SEQ ID No. 250)
SATPASAPYPLAGGGSSATPASAPYPLAGGGS-K(FITC)-amide
RP2-C
(SEQ ID No. 251)
SATPASAPYPLA-GGGS-K(Biotin)-C-amide

1. An isolated peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, and wherein the N-terminus of the peptide comprises a D-amino acid and/or includes a protecting group.

2. The peptide according to claim 1, wherein the peptide has a sequence with at least 60% homology to SEQ ID No. 1, or a functional portion or fragment thereof

3. The peptide according to claim 1 or claim 2, wherein the N-terminus of the peptide comprises a D-amino acid.

4. The peptide according to claim 1 or claim 2, wherein the the N-terminus of the peptide includes a protecting group.

5. The peptide according to claim 4, wherein the protecting group is selected from the group consiting of acetyl, benzoyl, benzyl, tert-butoxycarbonyl, carbobenzyloxy, p-methoxybenyl carbonyl, p-methoxybenzyl, 9-fluorenylmethyloxycarbonyl, 3,4-dimethoxybenzyl, p-methoxyphenyl, tosyl, and nosyl.

6. The peptide according to claim 5, wherein the protecting group is acetyl.

7. The peptide according to any one of claims 2 to 6, wherein the functional portion or fragment comprises at least 5 contiguous amino acids from SEQ ID No. 1 and shows at least 70% of the affinity of the peptide having at least 60% homology to SEQ ID No. 1 to hyaluronic acid and/or at least 70% of the affinity of the peptide having at least 60% homology to SEQ ID No. 1 to collagen.

8. The peptide according to any one of claims 2 to 7, wherein the peptide is a functional portion/fragment thereof having a sequence according to any of those shown in Table 1, and which shows at least 70% of the affinity of the peptide having at least 60% homology to SEQ ID No. 1 to hyaluronic acid and/or at least 70% of the affinity of the peptide having at least 60% homology to SEQ ID No. 1 to collagen.

9. A collagen or hyaluronic acid binding conjugate comprising a peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, wherein the N-terminus of the peptide comprises a D-amino acid and/or includes a protecting group, and a therapeutic or diagnostic agent, wherein the therapeutic or diagnostic agent is optionally bound to the peptide by means of a linker.

10. The collagen or hyaluronic acid binding conjugate according to claim 9, wherein the peptide has a sequence with at least 60% homology to SEQ ID No. 1, or a functional portion or fragment thereof.

11. The collagen or hyaluronic acid binding conjugate according to claim 9 or claim 10, wherein the protecting group is a nitrogen protecting group located on the nitrogen of the N-terminal amino acid of the peptide and is selected from the group consisting of acetyl, benzoyl, benzyl, tert-butoxycarbonyl, carbobenzyloxy, p-methoxybenyl carbonyl, p-methoxybenzyl, 9-fluorenylmethyloxycarbonyl, 3,4-dimethoxybenzyl, p-methoxyphenyl, tosyl, and nosyl.

12. The collagen or hyaluronic acid binding conjugate according to claim 11, wherein the protecting group is acetyl.

13. The collagen or hyaluronic acid binding conjugate according to any one of claims 9 to 12, wherein the therapeutic or diagnostic agent is covalently bound to the peptide.

14. The collagen or hyaluronic acid binding conjugate according to any one of claims 9 to 12, wherein the therapeutic or diagnostic agent is non-covalently bound to the peptide.

15. The collagen or hyaluronic acid binding conjugate according to claim 14, wherein the therapeutic or diagnostic agent is non-covalently bound to the peptide by means of a biotin-streptavidin complex.

16. The collagen or hyaluronic acid binding conjugate according to claim 15, wherein the peptide is covalently bound to the biotin moiety, optionally via a linker, and the therapeutic or diagnostic agent is covalently bound to the streptavidin moiety, optionally via a linker.

17. The collagen or hyaluronic acid binding conjugate according to claim 15, wherein the peptide is covalently bound to the streptavidin moiety, optionally via a linker, and the therapeutic or diagnostic agent is covalently bound to the biotin moiety, optionally via a linker.

18. The collagen or hyaluronic acid binding conjugate according to any one of claims 9 to 17, wherein the linker, when present, comprises a short-chain peptide, a polyethylene glycol oligomer, a C₁₋₂₀ alkylene group, a C₂₋₂₀ alkenylene group, maleimide and hydrazide functional groups separated by a C₁₋₂₀ alkylene or C₂₋₂₀ alkenylene group, or any combination thereof.

19. The collagen or hyaluronic acid binding conjugate according to claim 18, wherein the short-chain peptide of the linker comprises the amino acids glycine, serine, lysine, cysteine, glutamic acid and/or aspartic acid.

20. The collagen or hyaluronic acid binding conjugate according to any one of claims 9 to 19, wherein the linker, when present, is located at the C-terminus of the peptide.

21. The collagen or hyaluronic acid binding conjugate according to any one of claims 10 to 20, wherein the functional fragment comprises at least 5 contiguous amino acids from SEQ ID No. 1 and shows at least 70% of the affinity of the peptide having at least 60% homology to SEQ ID No. 1 to hyaluronic acid and/or at least 70% of the affinity of the peptide having at least 60% homology to SEQ ID No. 1 to collagen.

22. The collagen or hyaluronic acid binding conjugate according to any one of claims 10 to 20, wherein the peptide is a functional portion/fragment thereof having a sequence according to any of those shown in Table 1, and which shows at least 70% of the affinity of the peptide having at least 60% homology to SEQ ID No. 1 to hyaluronic acid and/or at least 70% of the affinity of the peptide having at least 60% homology to SEQ ID No. 1 to collagen.

23. The collagen or hyaluronic acid binding conjugate according to any one of claims 9 to 22, wherein the diagnostic agent comprises a fluorescent, luminescent, or radionuclide label.

24. The collagen or hyaluronic acid binding conjugate according to any one of claims 9 to 22, wherein the therapeutic agent is at least one selected from the group consisting of VEGF inhibitors, alpha2-adrenergic agonists, beta-adrenergic antagonists, Angiotensin II antagonists, ACE inhibitors, NSAIDs, antimalarials, corticosteroids, immune suppressants, monoclonal antibodies, retinoids, DMARDs, biologics, nitrates, prostaglandins, and endothelin antagonists.

25. A pharmaceutical composition comprising a peptide according to any one of claims 1 to 8, or a collagen or hyaluronic acid binding conjugate according to any one of claims 9 to 24, and at least one pharmaceutically acceptable excipient.

26. The pharmaceutical composition according to claim 25, further comprising at least one additional unconjugated therapeutic agent selected from the group consisting of VEGF inhibitors, alpha2-adrenergic agonists, beta-adrenergic antagonists, Angiotensin II antagonists, ACE inhibitors, NSAIDs, antimalarials, corticosteroids, immune suppressants, monoclonal antibodies, retinoids, DMARDs, biologics, nitrates, prostaglandins, and endothelin antagonists.

27. The peptide according to any one of claims 1 to 8, the collagen or hyaluronic acid binding conjugate according to any of claims 9 to 24, or the pharmaceutical composition according to claim 25 or claim 26, for use in therapy.

28. The peptide according to any one of claims 1 to 8, the collagen or hyaluronic acid binding conjugate according to any of claims 9 to 24, or the pharmaceutical composition according to claim 25 or claim 26, for use in the prophylaxis or treatment of age-related macular degeneration, diabetic retinopathy, diabetic macular oedema, retinal vein occlusion, retinopathy of prematurity, pathologic myopia macular oedema, macular telangiectasia, choroidal neovascularisation, uveitis, glaucoma, systemic lupus erythematosus, arthritis, rheumatoid arthritis, scleroderma, polymyositis, or dermatomyositis.

29. Use of an isolated peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, wherein the N-terminus of the peptide comprises a D-amino acid and/or includes a protecting group, for preparing a collagen or hyaluronic acid binding conjugate.

30. The use according to claim 29, wherein the peptide has a sequence with at least 60% homology to SEQ ID No. 1, or a functional portion or fragment thereof.

31. The use according to claim 30, wherein the functional portion or fragment comprises at least 5 contiguous amino acids from SEQ ID No. 1 and shows at least 70% of the affinity of the peptide having at least 60% homology to SEQ ID No. 1 to hyaluronic acid and/or at least 70% of the affinity of the peptide having at least 60% homology to SEQ ID No. 1 to collagen.

32. The use according to claim 30 or claim 31, wherein the peptide is a functional portion or fragment thereof having a sequence according to any of those shown in Table 1, and which shows at least 70% of the affinity of the peptide having at least 60% homology to SEQ ID No. 1 to hyaluronic acid and/or at least 70% of the affinity of the peptide having at least 60% homology to SEQ ID No. 1 to collagen.

33. An isolated peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, for use in the prophylaxis or treatment of ocular diseases or conditions, such as age-related macular degeneration, diabetic retinopathy, diabetic macular oedema, retinal vein occlusion, retinopathy of prematurity, pathologic myopia macular oedema, macular telangiectasia, choroidalneovascularisation, uveitis, or glaucoma.

34. The peptide for use according to claim 33, wherein the peptide has a sequence with at least 60% homology to SEQ ID No. 1, or a functional portion or fragment thereof.

35. The peptide for use according to claim 33 or claim 34, wherein the N-terminus of the peptide comprises a D-amino acid and/or includes a protecting group.

36. A collagen or hyaluronic acid binding conjugate comprising a peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, and a therapeutic or diagnostic agent, wherein the therapeutic or diagnostic agent is optionally bound to the peptide by means of a linker, for use in the prophylaxis or treatment of ocular diseases or conditions, such as age-related macular degeneration, diabetic retinopathy, diabetic macular oedema, retinal vein occlusion, retinopathy of prematurity, pathologic myopia macular oedema, macular telangiectasia, choroidal neovascularisation, uveitis, or glaucoma.

37. A method of detecting a hyaluronic acid binding substance, the method comprising providing a sample of hyaluronic acid, contacting the sample of hyaluronic acid with a test substance, and detecting the presence of binding between the test substance and the hyaluronic acid.

38. The method according to claim 37, wherein the hyaluronic acid is non-covalently bound to a solid support.

39. The method according to claim 38, wherein the solid support is an amine surface.

40. The method according to any one of claims 37 to 39, wherein bovine serum albumin is employed as a blocking agent and/or as a diluent.

41. The method according to any one of claims 37 to 40, wherein detection carried out using a biotinylated test substrate and streptavidin-horse radish peroxidase, with the addition of a peroxidase substrate.

42. A method of preventing or treating a condition associated with age-related macular degeneration, diabetic retinopathy, diabetic macular oedema, retinal vein occlusion, retinopathy of prematurity, pathologic myopia macular oedema, macular telangiectasia, choroidal neovascularisation, uveitis, glaucoma, systemic lupus erythematosus, arthritis, rheumatoid arthritis, scleroderma, polymyositis, or dermatomyositis, comprising administering to a subject in need thereof the peptide according to any one of claims 1 to 8, the collagen or hyaluronic acid binding conjugate according to any of claims 9 to 24, or the pharmaceutical composition according to claim 25 or claim 26.

43. A method of preventing or treating an ocular disease or condition, such as age-related macular degeneration, diabetic retinopathy, diabetic macular oedema, retinal vein occlusion, retinopathy of prematurity, pathologic myopia macular oedema, macular telangiectasia, choroidal neovascularisation, uveitis, or glaucoma, comprising administering to a subject in need thereof a peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids.

44. A method of preventing or treating an ocular disease or condition, such as age-related macular degeneration, diabetic retinopathy, diabetic macular oedema, retinal vein occlusion, retinopathy of prematurity, pathologic myopia macular oedema, macular telangiectasia, choroidal neovascularisation, uveitis, or glaucoma, comprising administering to a subject in need thereof a collagen or hyaluronic acid binding conjugate comprising a peptide comprising at least one motif having the amino acid sequence B¹-X₃₋₁₀-B², wherein B¹ and B² are identical or different and each is a basic amino acid and X₃₋₁₀ is a sequence of 3 to 10 identical or different non-acidic amino acids, and a therapeutic or diagnostic agent, wherein the therapeutic or diagnostic agent is optionally bound to the peptide by means of a linker.
